(19) United States
(12) Patent Application Publication
(10) Pub. No.: US 2024/0415964 A1
(43) Pub. Date: Dec. 19, 2024

(54) NATURAL KILLER CELLS FOR CHORDOMA THERAPY

(71) Applicant: ImmunityBio, Inc., San Diego, CA (US)

(72) Inventor: Patrick Soon-Shiong, San Diego, CA (US)

(21) Appl. No.: 18/705,950

(22) PCT Filed: Nov. 1, 2022
(86) PCT No.: PCT/US2022/048613

§ 371 Date: Apr. 29, 2024

Related U.S. Application Data
(60) Provisional application No. 63/274,908, filed on Nov. 2, 2021.

(51) Int. Cl. A61K 39/00; A61K 39/395; A61P 35/00

(52) U.S. Cl. CPC A61K 39/4613; A61K 39/3955; A61K 39/4631; A61K 39/464411; A61P 35/00; A61K 2039/505; A61K 2039/545

(57) ABSTRACT

Provided herein are methods and compositions for treating chordoma by preferentially targeting cancer stem cells than non-cancer stem cells in chordoma to effectively treat chordoma. In some embodiments, the chordoma tumors cells (preferentially the cancer stem cells in chordoma) are targeted by modified NK-92 cells (i.e., haNK® cells) that express CARs that comprise means for binding to the PD-L1 and another tumor-specific antigen (e.g., EGFR). In some embodiments, the chordoma tumor cells are targeted by using NK cells in combination with a means for binding to means for binding to the PD-L1 and/or a means for binding another tumor antigen (e.g., EGFR). Each of the treatment methods disclosed above may be implemented prior to, and/or concurrent with radio- and/or chemotherapy, and/or may be employed with immune therapy as is discussed in more detail below.

### RELATED APPLICATION

The application claims priority to U.S. Provisional Application No. 63/274,908, filed on Nov. 2, 2021. The entire content of the said provisional application is herein incorporated by reference for all purposes.

### FIELD

The field of the invention is modified immune competent cells for treating diseases, especially as it relates to high-affinity natural killer (haNK) cells and anti-PD-L1 compositions for treating chordoma.

### BACKGROUND

The background description includes information that may be useful in understanding the present invention. It is not an admission that any information provided herein is prior art relevant to the presently claimed invention or that any publication expressly or implicitly referenced is prior art.

Chordoma is a rare, slow-growing, locally invasive primary bone tumor thought to develop from embryonic notochord remnants, resulting in its predilection for the axial skeleton. The annual incidence of chordoma in the United States is roughly 300 cases per year (Heery et al. Oncol Ther 4, 35-51 (2016)). In adults, approximately half of these tumors arise in the sacrum, with the remainder occurring in the spheno-occipital region of the skull base (35%) and mobile spine (15%) (Chugh et al., Oncologist 12, 1344-1350 (2007). These anatomic regions are often in close proximity to critical neurovascular structures, thus posing technical challenges and morbidity to the current treatment mainstays of surgical resection and radiotherapy. Furthermore, chordomas are resistant to cytotoxic chemotherapy (Stacchiotti et al., Lancet Oncol 16, e71-83 (2015).). Challenges to conventional therapeutic approaches are evidenced by rates of local recurrence as high as 68% and metastases in up to 40% of cases (Chugh et al., Oncologist 12, 1344-1350 (2007), McPherson et al., J Neurosurg Spine 5, 277-280 (2006)). Median overall survival after diagnosis is approximately 5-7 years and decreases in patients with recurrent or metastatic disease (Chugh et al., Oncologist 12, 1344-1350 (2007)). Efforts to explore the application of immunotherapy in chordoma have led to clinical trials investigating targeted agents such as therapeutic vaccines (Heery et al., Cancer Immunol Res 3, 1248-1256 (2015); DeMaria et al., Oncologist 26, e847-e858 (2021)) and existing immunotherapeutic mainstays such as immune checkpoint blockade (NCT03173950, NCT02936102). However, these trials target chordoma patients with advanced disease and have yet to deliver durable responses. In the setting of recurrent and metastatic chordoma, survival rates are further compromised, and patients suffer increased morbidity from multiple rounds of surgical resection and radiation. Novel immunotherapy approaches hold promise to fill this unmet clinical need in treating chordoma.

Thus, while various treatment methods and compositions for chordoma are known in the art, all or almost all of them suffer from one or more disadvantages. There remains a need for improved compositions and methods for the treatment of chordoma.

### SUMMARY

Various objects, features, aspects, and advantages of the inventive subject matter will become more apparent from the following detailed description of preferred embodiments, along with the accompanying drawing figures in which like numerals represent like components

In one aspect, provided herein is a method of treating chordoma, the method comprising administering to a subject in need thereof a plurality of haNK® cells at a dosage effective to treat the chordoma, wherein the haNK® cells each comprise a first chimeric antigen receptor (CAR) comprising a means for binding PD-L1 and a second CAR comprising an amino acid sequence having at least 85% identity to a sequence selected from the group consisting of SEQ ID NOs: 6-20.

In another aspect, provided herein is a method of treating chordoma comprising: stimulating NK cells with an IL-15 superagonist; and administering to a subject in need thereof the NK cells and a means for binding PD-L1 and/or a means for binding EGFR.

In yet another aspect, provided herein is a kit for treating chordoma in a subject in need thereof. In some embodiments, the composition comprises (i) NK cells and (ii) means for binding PD-L1 and/or a means for binding EGFR. In some embodiments, the composition comprises a plurality of haNK® cells, wherein the haNK® cells each comprise a first chimeric antigen receptor (CAR) comprising a means for binding PD-L1 and a second CAR comprising an amino acid sequence having at least 85% identity to a sequence selected from the group consisting of SEQ ID NOs: 6-20.

### BRIEF DESCRIPTION OF THE DRAWING

FIG. 1A-1B show that anatomically distinct chordoma cell lines are susceptible to lysis by healthy donor natural killer (NK) cells. FIG. 1A shows that six chordoma cell lines; established from 3 from clival tumor patients (UM-Chor1, MUG-CC1, UM-Chor5), and three sacral tumor patients (JHC7, U-CH1, U-CH2) were analyzed by flow cytometry to quantify expression surface markers MHC-I/HLA-A, B, C, PD-L1, and EGFR. Cell surface expression of each marker is reported in % positive cells and MFI. FIG. 1B shows the chordoma cell killing by NK cells from 25 healthy donors as determined by ¹¹¹Indium-release (“In-release”) killing assays. Each data point represents a distinct healthy NK cell donor. All E:T ratios are 20:1. Statistical analyses were performed by Student's t-test. *P<0.05, **P<0.01, ***P<0.001, ****P<0.0001. The results shown are the means±SEM of at least five independent experiments.

FIG. 2A-2B show, that chordoma cell susceptibility to lysis by healthy donor NK cells is enhanced by N-601 (anti-PD-L1), N-803 (IL-15 superagonist), and combination treatment with N-601 and N-803. Six chordoma cell lines (UM-Chor1, MUG-CC1, UM-Chor5, JHC7, U-CH1, U-CH2) were used as targets for healthy donor NK cells in ¹¹¹In-release killing assays. FIG. 2A shows results of the N-601-mediated ADCC assays. The assays were performed by co-incubating chordoma cells with N-601 or isotype control antibody. NK cells were treated with anti-CD-16 antibodies where indicated. FIG. 2B shows the results of the combination treatment assays. The assays were performed by co-incubating chordoma cells with N-601 or isotype control antibody and treating NK cells with N-803 and/or anti-CD-16 antibody where indicated. All E:T ratios are 20:1. Statistical analyses were done by one-way ANOVA with Tukey's multiple comparisons test. *P≤0.05, **P≤0.01, ***P≤0.001, ****P≤0.0001. The results shown are the means±SEM of technical triplicate measurements and are representative of three independent experiments.

FIG. 3A shows that chordoma cell susceptibility to killing by healthy donor NK cells is enhanced by cetuximab (anti-EGFR). FIG. 3B shows that chordoma cell susceptibility to killing by healthy donor NK is further enhanced by combination treatment with N-803 (IL-15 superagonist) and cetuximab. Six chordoma cell lines (UM-Chor1, MUG-CC1, UM-Chor5, JHC7, U-CH1, U-CH2) were used as targets for healthy donor NK cells in In-release killing assays. FIG. 3A shows that Cetuximab-mediated ADCC assays were performed by co-incubating chordoma cells with Cetuximab or isotype control antibody. NK cells were treated with anti-CD-16 antibodies where indicated. FIG. 3B shows the results of combination treatment assays. The assays were performed by co-incubating chordoma cells with cetuximab or isotype control antibody and treating NK cells with N-803 and/or anti-CD-16 antibody where indicated. All E:T ratios are 20:1. Statistical analyses were done by one-way ANOVA with Tukey's multiple comparisons test. *P≤0.05, **P≤0.01, ***P≤0.001, ****P≤0.0001. The results shown are the means±SEM of technical triplicate measurements and are representative of three independent experiments.

FIG. 4 Doublet treatment of chordoma cells with N-601 (anti-PD-L1) and cetuximab (anti-EGFR) further enhances sensitivity to lysis by N-803 (IL-15 superagonist)-activated NK cells. Two chordoma cell lines (UM-Chor1, JHC7) were used as targets for healthy donor NK cells in ¹¹¹In-release killing assays. Chordoma cells were co-incubated with isotype control antibody, N-601, and/or cetuximab, and NK cells were treated with N-803 where indicated. All E:T ratios are 10:1. Statistical analyses were done by one-way ANOVA with Tukey's multiple comparisons test. *P≤0.05, **P≤0.01, ***P≤0.001, ****P≤0.0001. The results shown are the means±SEM of technical triplicate measurements and are representative of three independent experiments.

FIG. 5A-5C show chordoma patients' NK cells mediate significant lysis of chordoma cells and are enhanced with N-601 (anti-PD-L1), cetuximab (anti-EGFR), and N-803 (IL-15 superagonist). Two chordoma cell lines (UM-Chor1, JHC7) were used as targets for healthy donor NK cells or chordoma patient NK cells in ¹¹¹In-release killing assays. Combination treatment assays were performed by co-incubating chordoma cells with N-601 (FIGS. 5A and 5B), cetuximab (FIGS. 5A and 5C), or isotype control antibody where indicated. Healthy donor NK cells, treatment-naïve chordoma patient (“CP1(n)”) NK cells (FIG. 5A), or previously-treated chordoma patient (“CP2(io)”) NK cells (FIGS. 5B and 5C) were treated with N-803 where indicated and used as effectors. All E:T ratios are 20:1. Statistical analyses were done by one-way ANOVA with Tukey's multiple comparisons test. *P<0.05, **P<0.01, ***P<0.001, ****P<0.0001. Results shown are the means±SEM of technical triplicate measurements and are representative of two independent experiments with recurrent chordoma patients and one experiment with a treatment-naïve chordoma patient.

FIG. 6A-6B show that PD-L1 t-haNK cells induce lysis of chordoma cell lines through a PD-L1-mediated mechanism. Two chordoma cell lines (UM-Chor1, JHC7) were used as targets for PD-L1 t-haNK cells in ¹¹¹In-release killing assays. FIG. 6A shows the results of chordoma cells that were analyzed by flow cytometry for PD-L1 expression after 24-hour co-incubation with IFNy on the same day as killing assays. Cell surface expression of PD-L1 quantified by flow cytometry is reported in % positive cells and MFI. Bold values denote an increase of >30% relative to untreated control cells. FIG. 6B shows the results of chordoma cells co-incubated with IFNy or left untreated as controls for 24 hours before being used as targets for PD-L1 t-haNK cells. All E:T ratios are 20:1. Statistical analyses were done by one-way ANOVA with Tukey's multiple comparisons test. *P≤0.05, **P≤0.01, ***P≤0.001, ****P≤0.0001. The results shown are the means±SEM of technical triplicate measurements and are representative of three independent experiments.

FIG. 7A-7B show results of targeting chordoma CSCs through N-601 (anti-PD-L1)-mediated ADCC by N-803 (IL-15 superagonist)-activated NK cells. CellTrace Violet-stained UM-Chor1 cells were used as targets for healthy donor NK cells in a flow cytometry-based killing assay. FIG. 7A shows the results of identifying CSC and non-CSC subpopulations using a gating strategy. FIG. 7B shows that UM-Chor1 cells were co-incubated with N-601 or isotype control antibody, and NK cells were treated with N-803 where indicated. CSC and non-CSC cell death through N-601-mediated ADCC by untreated or N-803-treated NK cells were evaluated via flow cytometry using a live/dead fixable cell stain exclusion method. Flow cytometry was used to quantify the expression of HLA-A, B,C, MICA/B, B7-H6, and PD-L1 expression on CSC and non-CSC UM-Chor1 cells. Cell surface expression of each marker is reported in % positive cells and MFI. Bold values denote an increase of >20% when comparing CSC vs. non-CSC. All E:T ratios are 5:1. Statistical analyses were done by one-way ANOVA with Tukey's multiple comparisons test. *P≤0.05, **P≤0.01, ***P≤0.001, ****P≤0.0001. Results are shown in Table 4. These results are the means±SEM of technical quadruplicate measurements and are representative of two independent experiments.

FIG. 8 shows that chordoma cell lines express PD-L1 and EGFR with small subpopulations of PD-L1+/EGFR− cells. Six chordoma cell lines were analyzed by flow cytometry. Representative flow cytometric scatter plots of PD-L1, and EGFR co-expression are shown here.

FIG. 9 illustrates N-803-enhanced NK cells binding to ADCC-mediating antibodies on a chordoma cell, inducing tumor cell lysis.

FIG. 10 shows that previously treated chordoma patients' NK cells demonstrate enhanced cytotoxicity and are further enhanced with N-601 (anti-PD-L1), cetuximab (anti-EGFR), and N-803 (IL-15 superagonist).

FIG. 11 shows that the lysis of chordoma cells by PD-L1 t-haNK cells is not enhanced by ADCC-mediating antibodies (N-601, cetuximab) or N-803. UM-Chor1 cells were used as target cells for PD-L1 t-haNK cells in ¹¹¹In-release killing assays. Target cells were co-incubated with N-601 and/or cetuximab where indicated. PD-L1 t-haNK effector cells were treated with N-803 where indicated.

### DETAILED DESCRIPTION

### Overview

Provided herein are methods and compositions for treating chordoma by preferentially targeting cancer stem cells over non-cancer stem cells in chordoma.

In some embodiments, the chordoma tumors cells (preferentially the cancer stem cells in chordoma) are targeted by modified NK-92 cells (i.e., haNK® cells) that express CARs that comprise means for binding to PD-L1 and another tumor antigen (e.g., EGFR). Thus, in some embodiments, the methods and compositions comprise treating patients with haNK cells that express a first CAR comprising a means for binding PD-L1 and also express a second CAR comprising a means for binding a tumor-specific antigen that is not PD-L1 (a non-PD-L1 tumor-specific antigen). In some embodiments, the second CAR comprises an amino acid sequence having at least 85% sequence identity to any of SEQ ID NOs: 6-20.

In some embodiments, the chordoma tumor cells (preferentially the cancer stem cells in chordoma) are targeted by using NK cells in combination with a means for binding to PD-L1 and/or a means for binding another tumor antigen (e.g., EGFR). Thus, in some embodiments, the methods and compositions comprise administering to a chordoma patient NK cells and a means for binding to the PD-L1 (e.g., an anti-PD-L1 antibody) and/or a means for binding another tumor antigen, e.g., an antibody binds to the EGFR. Each of the treatment methods disclosed above may be implemented prior to and/or concurrent with, radio- and/or chemotherapy, and/or may be employed with immune therapy, as is discussed in more detail below.

Because cancer stem cells are implicated in chordoma's resistant and recurrent nature, the methods and compositions disclosed herein, which preferentially target these cancer stem cells, offer an effective treatment option for chordoma patients.

Unless defined otherwise, all technical and scientific terms used herein have the same meaning as commonly understood by one of ordinary skill in the art.

In this specification and in the claims that follow, reference will be made to several terms that shall be defined to have the following meanings:

The terminology used herein is to describe particular embodiments only and is not intended to be limiting. As used herein, the singular forms “a,” “an,” and “the” are intended to include the plural forms as well, unless the context clearly indicates otherwise. Thus, for example, reference to “a natural killer cell” includes a plurality of natural killer cells.

All numerical designations, e.g., pH, temperature, time, concentration, amounts, and molecular weight, including ranges, are approximations that are varied (+) or (−) by increments of 0.1 or 1.0, where appropriate. It is to be understood, although not always explicitly stated, that all numerical designations may be preceded by the term “about.”

As used herein, “+” when used to indicate the presence of a particular cellular marker, means that the cellular marker is detectably present in fluorescence-activated cell sorting over an isotype control; or is detectable above background in quantitative or semi-quantitative RT-PCR.

As used herein, “−,” when used to indicate the presence of a particular cellular marker, means that the cellular marker is not detectably present in fluorescence-activated cell sorting over an isotype control; or is not detectable above background in quantitative or semi-quantitative RT-PCR.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

It is also to be understood, although not always explicitly stated, that the reagents described herein are merely exemplary and that equivalents of such are known in the art.

The term “chimeric antigen receptor” (CAR), as used herein, refers to an extracellular antigen-binding domain that is fused to an intracellular signaling domain. CARs can be expressed in T cells or NK cells to increase cytotoxicity. In general, the extracellular antigen-binding domain is a scFv that is specific for an antigen found on a cell of interest. A CAR-expressing haNK® cell is targeted to cells expressing certain antigens on the cell surface, based on the specificity of the scFv domain. The scFv domain can be engineered to recognize any antigen, including tumor-specific antigens and virus-specific antigens. For example, PD-L1 CAR recognizes PD-L1, a cell surface marker expressed by some tumors.

The term “tumor-specific antigen” as used herein refers to antigens that are present on a cancer or neoplastic cell but not detectable on a normal cell derived from the same tissue or lineage as the cancer cell. Tumor-specific antigens, as used herein, also refers to tumor-associated antigens, that is, antigens that are expressed at a higher level on a cancer cell as compared to a normal cell derived from the same tissue or lineage as the cancer cell.

As used herein, the term “target,” when referring to targeting of a tumor or tumor cell, refers to the ability of NK-92® cells to recognize and kill a tumor cell (i.e., target cell). The term “targeted” in this context refers, for example, to the ability of a CAR expressed by the haNK® cell to recognize and bind to a cell surface antigen expressed by the tumor.

The term “antibody” refers to an intact immunoglobulin of any isotype, or a fragment thereof that can compete with the intact antibody for specific binding to the target antigen, and includes chimeric, humanized, fully human, and bispecific antibodies. An intact antibody generally comprises at least two full-length heavy chains and two full-length light chains, but in some instances can include fewer chains such as antibodies naturally occurring in camelids which can comprise only heavy chains. Antibodies can be derived solely from a single source, or can be “chimeric,” such that different portions of the antibody are derived from two different antibodies. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also includes peptibodies.

The term “means for binding” refers to compositions and methods used to effect binding of a polypeptide. For example, a means for binding a PD-L1 polypeptide include, for example, using an anti-PD-L1 antibody, a fragment thereof that is capable of binding the PD-L1 polypeptide, in binding the PD-L1 polypeptide.

The term “subject” refers to a human or a non-human animal, including a mammal, such as a cat, a dog, a cow, a horse, a pig, a sheep, or a goat. In some embodiments, a subject is a patient in need of treatment for a disease described herein.

“Optional” or “optionally” means that the subsequently described event or circumstance can or cannot occur, and that the description includes instances where the event or circumstance occurs and instances where it does not.

The term “comprising” means that the compositions and methods include the recited elements but not to exclude others. “Consisting essentially of,” when used to define compositions and methods, s hall mean excluding other elements of any essential significance to the combination. For example, a composition consisting essentially of the elements defined herein would not exclude other elements that do not materially affect the basic and novel characteristic(s) of the embodiments disclosed herein. “Consisting of” means excluding more than a trace amount of other ingredients and substantial method steps. Embodiments defined by each of these transition terms are within the scope of the disclosure.

As used herein, the terms “cytotoxic” and “cytolytic”, when used to describe the activity of effector cells such as NK cells, are intended to be synonymous. In general, cytotoxic activity relates to killing of target cells by any of a variety of biological, biochemical, or biophysical mechanisms. Cytolysis refers more specifically to activity in which the effector lyses the plasma membrane of the target cell, thereby destroying its physical integrity. This results in the killing of the target cell. Without wishing to be bound by theory, it is believed that the cytotoxic effect of NK cells is due to cytolysis.

The term “kill” with respect to a cell/cell population is directed to include any type of manipulation that will lead to the death of that cell/cell population.

The term “cytokine” or “cytokines” refers to the general class of biological molecules which effect cells of the immune system. Exemplary cytokines include but are not limited to FLT3 ligand, interferons and interleukins (IL), in particular IL-2, IL-12, IL-15, IL-18 and IL-21.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. The term “administering” or “administration” of a monoclonal antibody or a natural killer cell to a subject includes any route of introducing or delivering the antibody or cells to perform the intended function. Administration can be carried out by any route suitable for the delivery of the cells or monoclonal antibody. Thus, delivery routes can include intravenous, intramuscular, intraperitoneal, or subcutaneous delivery. In some embodiments the modified haNK® cells are administered directly to the tumor, e.g., by injection into the tumor. In some embodiments the modified haNK® cells described herein are administered parenterally, e.g., by injection, infusion or implantation (subcutaneous, intravenous, intramuscular, intravesicularly, or intraperitoneal).

The term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “therapeutically effective amount” or “effective amount” refers to the amount required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “immune cells” refers to cells of hematopoietic origin that are involved in the specific recognition of antigens. Immune cells include antigen presenting cells (APCs), such as dendritic cells or macrophages, B cells, T cells, natural killer cells, myeloid derived suppressor cells (MDSC), myeloid cells, such as monocytes, macrophages, eosinophils, mast cells, basophils, and granulocytes.

Titles or subtitles may be used in the specification for the convenience of a reader, which are not intended to influence the scope of the present disclosure. Additionally, some terms used in this specification are more specifically defined below.

Option I. Treating Chordoma with haNK® Cells Expressing CARS

In some embodiments, provided herein is a method of treating chordoma comprising administering to a subject in need thereof a plurality of haNK® cells at a dosage effective to treat the chordoma. The haNK® cells each comprises a first chimeric antigen receptor (CAR) comprising a means for binding PD-L1 and a second chimeric antigen comprising a means for binding a tumor-specific antigen that is not PD-L1. In some embodiments, the second CAR comprises a polypeptide having at least 85% amino acid sequence identity to a sequence selected from the group consisting of SEQ ID NOs: 6-20.

The haNK® cells disclosed herein are derived from NK-92® cells. NK-92® is a cytolytic cancer cell line which was discovered in the blood of a subject suffering from a non-Hodgkins lymphoma and then immortalized in vitro, as described in Gong et al. (Leukemia, April; 8(4): 652-8 (1994)), rights to which are owned by ImmunityBio. NK-92® cells are derived from NK cells, but lack the major inhibitory receptors that are displayed by normal NK cells, while retaining the majority of the activating receptors. NK-92® cells do not, however, attack normal cells nor do they elicit an unacceptable immune rejection response in humans. Characterization of the NK-92® cell line is disclosed in WO 1998/049268 and U.S. Patent Application Publication No. 2002-0068044. NK-92® cells have been evaluated as a therapeutic agent in the treatment of certain cancers.

For purposes of this disclosure and unless indicated otherwise, the term “NK-92®” is intended to refer to the original NK-92® cell lines as well as NK-92® cell lines, clones of NK-92® cells, and NK-92® cells that have been modified (e.g., by introduction of exogenous genes). NK-92® cells and exemplary and non-limiting modifications thereof are described in U.S. Pat. Nos. 7,618,817; 8,034,332; 8,313,943; 9,181,322; 9,150,636; and published U.S. application Ser. No. 10/008,955, all of which are incorporated herein by reference in their entireties, and include wild type NK-92®, NK-92®-CD16, NK-92®-CD16-γ, NK-92®-CD16-ζ, NK-92®-CD16(F176V), NK-92® MI, and NK-92® CI. NK-92® cells are known to persons of ordinary skill in the art, to whom such cells are readily available from NantKwest, Inc.

As used herein, the term “aNK® cells” refers to unmodified natural killer cells derived from the highly potent unique cell line described in Gong et al. (Leukemia, April; 8(4): 652-8 (1994)), rights to which are owned by ImmunityBio.

As used herein, “haNK® cells” refers to NK-92® cells that are modified and/or selected to express CD16 on the cell surface (hereafter, “CD16+haNK® cells” or “haNK® cells”). In some embodiments, the haNK® cells are further engineered to comprises a CAR comprising a means to bind to PD-L1 (a PD-L1 CAR) and optionally one or more other CARS, as further described below. haNK® cells that comprise a means to bind PD-L1 are referred to as PD-L1 t-haNK® cells, which was discussed in the Examples of this application. In some embodiments, the haNK® cells are further modified to have reduced or abolished expression of at least one inhibitory receptor (KIR), which renders such cells constitutively activated (via lack of or reduced inhibition).

### CAR

Chimeric antigen receptors (CARs) disclosed herein are typically introduced to be expressed on the surface of immune cells such as T cells and NK cells. One of the function of these CARs is to bind to tumor-specific antigens on cell surface. These tumor-specific antigens are typically expressed in neoplastic or tumor cells, but not in normal cells; or they are expressed in neoplastic cells at levels substantially above those found in normal cells. Tumor-specific antigens have been used as targets for CAR-expressing immune cells in cancer immunotherapies.

A CAR typically comprises a single-chain variable fragment (scFv) linked to at least one intracellular signaling domain. The scFv recognizes and binds an antigen on the target cell (e.g., a cancer cell) and triggers effector cell activation. The signaling domains contain immunoreceptor tyrosine-based activation domains (ITAMs) that are important for intracellular signaling by the receptor.

The present disclosure provides haNK® cells that have been engineered to express at least a chimeric antigen receptor (CAR), e.g., a PD-L1 CAR, on the cell surface. A CAR combine an extracellular antigen-recognizing part (usually derived from the variable domain of a specific antibody) and an intracellular signaling domain (either single or with additional co-stimulatory elements) that can trigger a cytolytic response once a specific antigen is recognized. There are multiple types of CARs, which all can be used in the application. The first generation of CARs contains one cytoplasmic signaling domain. The signaling domain can be from e.g., the Fc epsilon receptor gamma (FcεRIγ) which contains one ITAM, or from CD3ζ, which contains three ITAMs. It is believed that CD3ζ CARs are more efficient at tumor eradication than FcεRIγ CARs. See, e.g., Haynes, et al. 2001, J. Immunology 166:182-187; Cartellieri, et al. 2010, J. Biomed and Biotech, Vol. 2010, Article ID 956304. The second and third generation CARs combine multiple signaling domains, e.g., the cytoplasmic signaling domain of CD3ζ and costimulatory signaling domains, such as CD28/CD134/CD137/ICOS and CD28/CD134 to a single CAR to promote the activation and proliferation of the haNK® cells. Thus, in some embodiments, the CAR expressed by haNK® comprises a hinge region from CD8, and/or a transmembrane domain of CD28. In some embodiments, the CAR comprises a cytoplasmic signaling domain of FcεRIγ. In some embodiments, the CAR comprises the cytoplasmic signaling domain of CD3ζ. Examples of the hinge region, the transmembrane domain of CD28 and the cytoplasmic signaling domain of FcεRIγ or CD3ζ are disclosed in U.S. Provisional application No. 62/674,936, the entire content of which is herein incorporated by reference.

Disclosed herein are haNK® cells comprising a CAR comprising a means for binding Programmed death-ligand 1 (PD-L1). PD-L1 is constitutively expressed and induced in tumor cells, tumor-associated macrophages (TAMs), and myeloid-derived suppressor cell (MDSCs). See, Kuang et al., J. Exp. Med. 2009; 206:1327-1337. In some embodiments, PD-L1 is a human PD-L1. In some embodiments, the PD-L1 CAR comprises a scFv having an amino acid sequence set forth as SEQ ID NO: 5. In some embodiments, the PD-L1 CAR comprises SEQ ID NO: 27.

In some embodiments, the haNK® cell comprise one or more additional CARs that binds to other tumor-specific antigens. These tumor-specific antigens include, without limitation, EGFR, CD19, CD20, NKG2D ligands, CS1, GD2, CD138, EpCAM, EBNA3C, GPA7, CD244, CA-125, MUC-1, ETA, MAGE, CAGE, BAGE, HAGE, LAGE, PAGE, NY-SEO-1, GAGE, CEA, CD52, CD30, MUC5AC, c-Met, FAB, WT-1, PSMA, NY-ESO1, AFP, CSPG-4, IGF1-R, Flt-3, CD276, CD123, PD-L1, BCMA, CD33, 41BB, CTAG1B, and CD33. Additional non-limiting tumor-associated antigens, and the malignancies associated therewith, can be found in Table 1. Non-limiting examples of tumor-specific antigens are also in US2013/0189268; WO 1999024566 A1; U.S. Pat. No. 7,098,008; and WO 2000020460 A1, each of which is incorporated herein by reference in its entirety.

TABLE 1
Tumor-Specific Antigens and Associated Malignancies

| Target antigen | Associated malignancy |
|---|---|
| α-Folate receptor | Ovarian cancer |
| CAIX | Renal cell carcinoma |
| CD19 | B-cell malignancies |
| | CLL |
| | B-ALL |
| | ALL; ALL post-HSCT |
| | Lymphoma; Refractory |
| | Follicular Lymphoma; |
| | B-NHL |
| | Leukemia |
| | B-cell malignancies; B-cell |
| | malignancies post-HSCT |
| | B-lineage lymphoid malignancies |
| | post-UCBT |
| | B-cell malignancies, CLL, B-NHL |
| CD19/CD20 | Lymphoblastic leukemia |
| CD20 | Lymphomas |
| | B-cell malignancies |
| | B-cell lymphomas |
| | Mantle cell lymphoma |
| | indolent B-NHL |
| | Leukemia |
| CD22 | B-cell malignancies |
| CD30 | Lymphomas; Hodgkin lymphoma |
| CD33 | AML |
| CD44v7/8 | Cervical carcinoma |
| CD138 | Multiple myeloma |
| CD244 | Neuroblastoma |
| CEA | Breast cancer |
| | Colorectal cancer |
| CS1 | Multiple myeloma |
| EBNA3C | EBV positive T cells |
| EGP-2 | Multiple malignancies |
| EGP-40 | Colorectal cancer |
| EpCAM | Breast carcinoma |
| erb-B2 | Colorectal cancer |
| | Breast and others |
| | Prostate cancer |
| erb-B 2,3,4 | Breast and others |
| FBP | Ovarian cancer |
| Fetal acetylcholine receptor | Rhabdomyosarcoma |
| GD2 | Neuroblastoma |
| GD3 | Melanoma |
| GPA7 | Melanoma |
| Her2 | Breast carcinoma |
| | Ovarian cancer |
| | Tumors of epithelial origin |
| Her2/neu | Medulloblastoma |
| | Lung malignancy |
| | Advanced osteosarcoma |
| | Glioblastoma |
| IL-13R-a2 | Glioma |
| | Glioblastoma |
| | Medulloblastoma |
| KDR | Tumor neovasculature |
| k-light chain | B-cell malignancies |
| | (B-NHL, CLL) |
| LeY | Carcinomas |
| | Epithelial derived tumors |
| L1 cell adhesion molecule | Neuroblastoma |
| MAGE-A1 | Melanoma |
| Mesothelin | Various tumors |
| MUC1 | Breast, Ovary |
| NKG2D ligands | Various tumors |
| Oncofetal antigen (h5T4) | Various tumors |
| PSCA | Prostate carcinoma |
| PSMA | Prostate/tumor vasculature |
| TAA targeted by mAb IgE | Various tumors |
| TAG-72 | Adenocarcinomas |
| VEGF-R2 | Tumor neovasculature |

In some embodiments, a polynucleotide encoding a PD-L1 CAR or any CARs disclosed herein can be mutated to alter the amino acid sequence encoding for CAR without altering the function of the CAR. For example, polynucleotide substitutions leading to amino acid substitutions at “non-essential” amino acid residues can be made in these CARS, for example in the scFv portion of the CARs.

Conservative substitutions in any of the CARs whereby an amino acid of one class is replaced with another amino acid of the same class, fall within the scope of the disclosed variants as long as the substitution does not materially alter the activity of the polypeptide. Conservative substitutions are well known to one of skill in the art. Non-conservative substitutions that affect (1) the structure of the polypeptide backbone, such as a β-sheet or α-helical conformation, (2) the charge, (3) the hydrophobicity, or (4) the bulk of the side chain of the target site can modify polypeptide function or immunological identity. Non-conservative substitutions entail exchanging a member of one of these classes for another class. Substitutions may be introduced into conservative substitution sites or more preferably into non-conserved sites.

In examples, variant polypeptides are produced using methods known in the art such as oligonucleotide-mediated (site-directed) mutagenesis, alanine scanning, and PCR mutagenesis. Site direct mutagenesis (Carter, 1986; Zoller and Smith, 1987), cassette mutagenesis, restriction selection mutagenesis (Wells et al., 1985) or other known techniques can be performed on the cloned DNA to produce variants (Ausubel, 2002; Sambrook and Russell, 2001).

In some embodiments, a CAR polypeptide expressed by a haNK® cell disclosed shares at least 85%, 90, 91, 92, 93, 94, 95, 96, 97, 98, or 99% amino acid sequence identity to a canonical CAR polypeptide disclosed herein. In some embodiments, the haNK® may express a PD-L1 CAR comprising an scFv of an anti-PD-L1 antibody and the scFV has an amino acid sequence that shares at least 85%, 90, 91, 92, 93, 94, 95, 96, 97, 98, or 99% identity to SEQ ID NO: 5. In some embodiments, the haNK® may express a PD-L1 CAR comprising an amino acid sequence that shares at least 85%, 90, 91, 92, 93, 94, 95, 96, 97, 98, or 99% identity to SEQ ID NO: 27.

In some embodiments, the CAR comprises an scFv having an amino acid sequence selected from the group consisting of SEQ ID NOs: 6-20. In some embodiments, the CAR comprises an scFv sharing at least 85%, at least 90%, at least 95%, at least 98% amino acid sequence identity with any one of SEQ ID NOs: 6-20.

In some embodiments, an epitope tag peptide, such as FLAG, myc, polyhistidine, or V5 can be added to the amino terminal domain of the polypeptide to assist in cell surface detection by using anti-epitope tag peptide monoclonal or polyclonal antibodies.

Option II. Treating Chordoma with NK Cells

Also disclosed herein is a method of treating chordoma using NK cells. As used herein, the term “NK cells” refer to unmodified NK cells, for example, those isolated from whole blood or cultivated from precursor or stem cells using methods known in the art. In some embodiments, the method comprises stimulating the NK cells with an IL-15 superagonist, and administering to the subject in need thereof the NK cells and a means for binding PD-L1 and/or a means for binding EGFR. In some embodiments, the NK cells are autologous NK cells, i.e., NK cells obtained from the same subject who will receive the NK cells during treatment. In some embodiments, the NK cells are allogenic NK cells, i.e., NK cells obtained from a subject that is different from the recipient of these NK cells. Still further, it is contemplated that the NK cells may be HLA matched NK cells, which may be primary cells, NK cells differentiated from upstream stem or progenitor cells, or cultured NK cells.

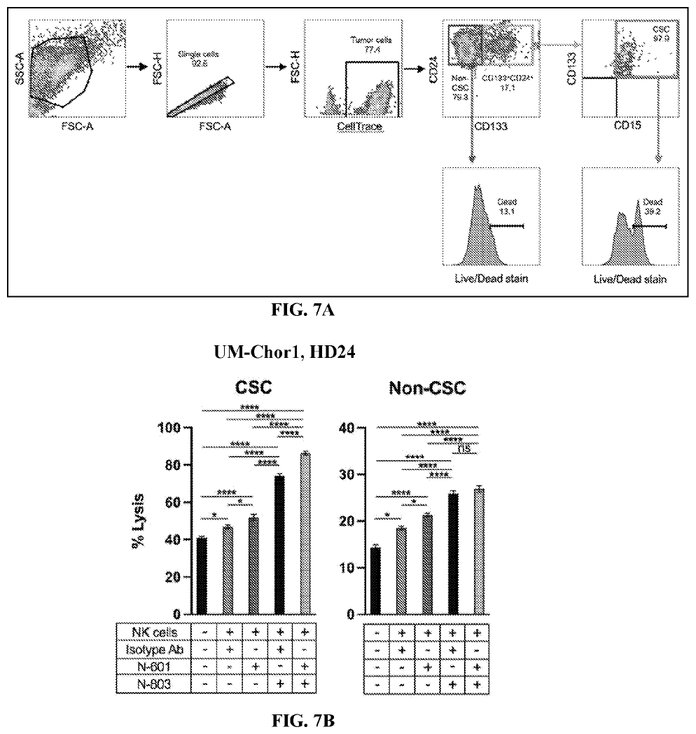

As shown in Example 9, CSCs were susceptible to anti-PD-L1 antibody (N-601)-mediated ADCC. This is evidenced by an increase in cell death in both CSC and non-CSC populations compared to isotype control (P≤0.05; FIG. 7B). Likewise, activating NK cells with N-803 improved their cytotoxicity against both CSCs and non-CSCs when compared to untreated controls (P≤0.0001). A combinatory approach with N-601 and N-803 further enhanced NK cell lysis of CSC cells (P≤0.0001), while cell death of non-CSCs by N-803 pre-treated NK cells remained the same with or without N-601 (P=0.46) (FIG. 7B). These results indicate that the CSCs are much more susceptible to the combination therapy of NK cells plus an IL-15 superagonist plus anti-PD-L1 antibody, and killing these CSCs using the combination therapy will greatly improve the success rate of treating chordoma.

Thus also provided herein is a method of treating chordoma comprising administering to a subject in need thereof the NK cells and a means for binding PD-L1 (e.g., an anti-PD-L1 antibody) and/or a means for binding EGFR (e.g., an anti-EGFR antibody). In some embodiments, the method further comprises administering IL-15 or an IL-15 superagonist, such as N-803 to activate the NK cells, as further described below.

### Chordoma Cancer Stem Cells

Cancer stem cells (CSC), or tumor-initiating cells (TIC), are a subpopulation of malignant cells that can drive tumorigenesis and disease relapse (17-20). This cell type is believed to be involved in tumor heterogeneity and resistance to chemotherapy and radiation. CSCs have been identified in myeloid malignancies, glioblastoma, and cancers of the breast, colon, pancreas, and skin (Dalerba et al., N Engl J Med 374, 211-222 (2016)). A CSC expresses surface markers CD15, CD24, and CD133 (8, 22); in contrast a non-CSC cell lacks expression of at least one of the CD15, CD24, or CD133.

The methods and compositions disclosed herein preferentially target CSCs in chordoma, which can effectively treat chordoma. As used herein, the term “preferentially target,” refers to under the same conditions, the therapy kills more one type of cells than other. The haNK® cells disclosed herein preferentially target chordoma cancer stem cells than chordoma non-CSCs, i.e., they kill at least 20%, at least 30%, at least 40%, or at least 50% more of chordoma cancer stem cells than chordoma non-CSCs when under the same conditions under which these cells can be lysed by these haNK® cells. As shown in Example 9 and Table 4, CSCs and non-CSCs showed comparable expression of MICA/B, a ligand for the activating receptor NKG2D, and compHLA-A,B,C/MHC class I, which binds NK inhibitory receptors. However, CSCs expressed B7-H6 (a ligand for the NK activating receptor NKp30) at higher levels than the non-CSCs. In addition, these chordoma CSCs have elevated expression of PD-L1, both as % positive cells and MFI (>1000% difference in % positive cells, >20% difference in MFI) (Table 4). This upregulation of an NK-activating ligand and PD-L1 in the UM-Chor1 CSC population may sensitize CSCs to lysis by NK cells expressing CARs that can bind to PD-L1, or haNK® cells that express a CARs that binds to PD-L1 and other CARS as disclosed above.

TABLE 4
Differential expression profile between CSCs and non-CSCs

| | HLA-A, B, C | | MICA/B | | B7-H6 | | PD-L1 | |
|---|---|---|---|---|---|---|---|---|
| | % positive | MFI | % positive | MFI | % positive | MFI | % positive | MFI |
| CSC | 99.8 | 15859 | 83.9 | 392 | 72.0 | 350 | 63.8 | 1033 |
| Non-CSC | 99.5 | 12295 | 75.8 | 383 | 6.9 | 25 | 6.1 | 835 |

Methods for assessing various NK cells or the haNK cells in killing tumor cells are known. Non-limiting examples of such methods include the flow-based NK cell killing assay and the Indium based NK cell killing assay, which are further described in Example 1.

### PD-L1 Antibodies

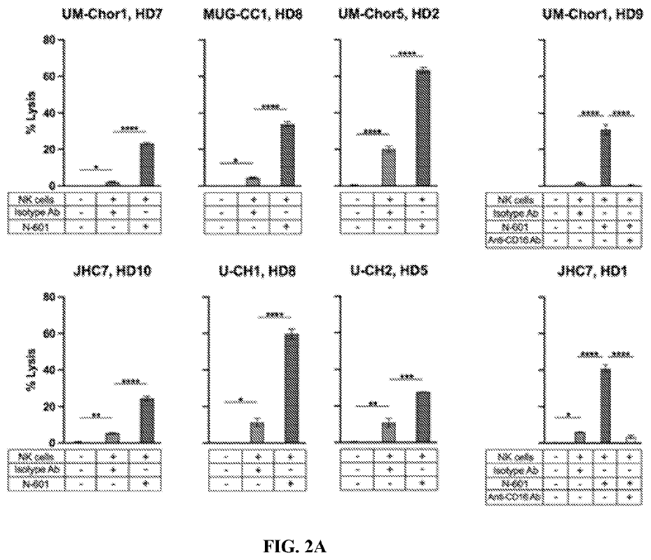

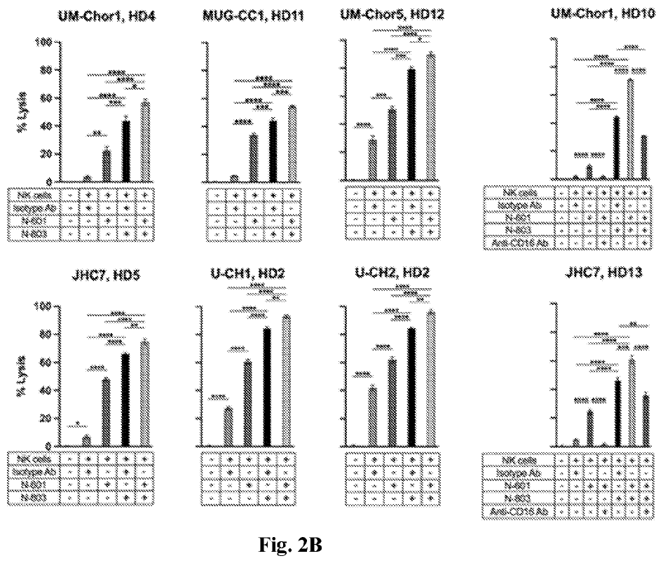

Various embodiments of the methods and compositions in this disclosure involves means for binding PD-L1 expressed on the tumor cell surface. In some embodiments, the means for binding PD-L1 is through a PD-L1 antibody. In some embodiments, the antibody is a monoclonal antibody. In some embodiments, the means for binding PD-L1 is through a PD-L1 CAR expressed on the haNK® cells disclosed herein. A PD-L1 CAR typically comprises an extracellular portion that recognizes the PD-L1 on the cell surface. This extracellular antigen-recognizing portion can be derived from any antibody's variable domain that demonstrates specific binding to PD-L1. Non-limiting examples of PD-L1 antibodies include Avelumab, Atezolizumab, Durvalumab, and N-601. Atezolizumab is a fully humanized IgG1 approved for urothelial carcinoma and non-small cell lung cancer. Avelumab is a fully human IgG1 antibody approved for treating metastatic merkel-cell carcinoma. Durvalumab is a fully human IgG1 antibody approved for treating urothelial carcinoma and unresectable non-small cell lung cancer. N-601 is an anti-PD-L1 monoclonal antibody (mAb) and structural analog of avelumab that has not been previously investigated. To date, Avelumab (anti-PD-L1) is the only immune checkpoint inhibitor that promotes antibody-dependent cellular cytotoxicity (ADCC) with NK cells. Avelumab has also demonstrated efficacy in preclinical models of chordoma (Fujii et al., Oncotarget 7, 33498-33511 (2016)). FIG. 2A shows that N-601 increased NK cell lysis of all six chordoma cell lines tested significantly.

### FC Receptors

The haNK® cells disclosed herein express at least one Fc receptor on the cell surface. Fc receptors bind to the Fc portion of antibodies. Suitable Fc receptors are known and differ according to their preferred ligand, affinity, expression, and effect following binding to the antibody. Illustrative Fc receptors are shown in Table 2.

TABLE 2
Illustrative Fc receptors

| | Principal | Affinity | | Effect following |
|---|---|---|---|---|
| | antibody | for | | binding to |
| Receptor name | ligand | ligand | Cell distribution | antibody |
| FcγRI (CD64) | IgG1 and | High | Macrophages | Phagocytosis |
| | IgG3 | (Kd~10⁻⁹M) | Neutrophils | Cell activation |
| | | | Eosinophils | Activation of respiratory |
| | | | Dendritic cells | burst |
| | | | | Induction of microbe |
| | | | | killing |
| FcγRIIA (CD32) | IgG | Low | Macrophages | Phagocytosis |
| | | (Kd > | Neutrophils | Degranulation (eosinophils) |
| | | 10⁻⁷M) | Eosinophils | |
| | | | Platelets | |
| | | | Langerhans cells | |
| FcγRIIB1 (CD32) | IgG | Low | B Cells | No phagocytosis |
| | | (Kd > | Mast cells | Inhibition of cell activity |
| | | 10⁻⁷M) | | |
| FcγRIIB2 (CD32) | IgG | Low | Macrophages | Phagocytosis |
| | | (Kd > | Neutrophils | Inhibition of cell activity |
| | | 10⁻⁷M) | Eosinophils | |
| FcγRIIIA (CD16a) | IgG | Low | NK cells | Induction of antibody- |
| | | (Kd > | Macrophages (certain | dependent cell-mediated |
| | | 10⁻⁶M) | tissues) | cytotoxicity (ADCC) |
| | | | | Induction of cytokine |
| | | | | release by macrophages |
| FcγRIIIB (CD16b) | IgG | Low | Eosinophils | Induction of microbe |
| | | (Kd > | Macrophages | killing |
| | | 10⁻⁶M) | Neutrophils | |
| | | | Mast cells | |
| | | | Follicular dendritic | |
| | | | cells | |
| FcεRI | IgE | High | Mast cells | Degranulation |
| | | (Kd~10⁻¹⁰M) | Eosinophils | Phagocytosis |
| | | | Basophils | |
| | | | Langerhans cells | |
| | | | Monocytes | |
| FcεRII (CD23) | IgE | Low | B cells | Possible adhesion molecule |
| | | (Kd > | Eosinophils | IgE transport across the |
| | | 10⁻⁷M) | Langerhans cells | human intestinal epithelium |
| | | | | Positive-feedback |
| | | | | mechanism to enhance |
| | | | | allergic sensitization (B |
| | | | | cells) |
| FcαRI (CD89) | IgA | Low | Monocytes | Phagocytosis |
| | | (Kd > | Macrophages | Induction of microbe |
| | | 10⁻⁶M) | Neutrophils | killing |
| | | | Eosinophils | |
| Fcα/μR | IgA and IgM | High for | B cells | Endocytosis |
| | | IgM, | Mesangial cells | Induction of microbe |
| | | Mid for | Macrophages | killing |
| | | IgA | | |
| FcRn | IgG | | Monocytes | Transfers IgG from a |
| | | | Macrophages | mother to fetus through the |
| | | | Dendritic cells | placenta |
| | | | Epithelial cells | Transfers IgG from a |
| | | | Endothelial cells | mother to an infant in milk |
| | | | Hepatocytes | Protects IgG from |
| | | | | degradation |

In some embodiments, the Fc receptor is CD16. For purposes of this disclosure, specific amino acid residues of CD16 are designated with reference to SEQ ID NO:2 or to SEQ ID NO: 1, which differs at one position relative to SEQ ID NO:1. Thus, an amino acid residue “at position 158” of a CD16 polypeptide is the amino acid residue that corresponds to position 158 of SEQ ID NO:2 (or SEQ ID NO:1), when the CD16 polypeptide and SEQ ID NO:2 are maximally aligned. In some embodiments, haNK® cells express a human CD16 that has a phenylalanine at position 158 of the mature form of the protein, e.g., SEQ ID NO:1. In typical embodiments, haNK® cells express a high-affinity form of human CD16 having a valine at position 158 of the mature form of the protein, e.g., SEQ ID NO:2. Position 158 of the mature protein corresponds to position 176 of the CD16 sequence that includes the native signal peptide. Thus, in one embodiment, the Fc receptor comprises FcγRIII-A (CD16). In some embodiments, the haNK® cells express an Fc receptor encoding a polypeptide having at least 90% sequence identity with SEQ ID NO:1 (FcγRIII-A or CD16 having a phenylalanine at position 158 (F-158); or at least 90% identity to SEQ ID NO:2 (CD16 having a valine at position 158 (F158V), higher affinity form).

In some embodiments, the CD16 polynucleotide encodes a polypeptide having at least 70%, 80%, 90%, or 95% identity to SEQ ID NO:1 or SEQ ID NO:2. In some embodiments, the polynucleotide encodes a polypeptide having at least 70% 80%, 90%, or 95% identity to SEQ ID NO:2 and comprises a valine at position 158 as determined with reference to SEQ ID NO:2. In some embodiments the polynucleotide encodes SEQ ID NO:2. In some embodiments, a CD16 polynucleotide encodes an extracellular domain of CD16 with or without the signal sequence, or any other fragment of a full-length CD16, or a chimeric receptor encompassing at least partial sequence of CD16 fused to an amino acid sequence of another protein.

In some embodiments, homologous CD16 polynucleotides may be about 150 to about 700, about 750, or about 800 polynucleotides in length, although CD16 variants having more than 700 to 800 polynucleotides are within the scope of the disclosure.

Homologous polynucleotide sequences include those that encode polypeptide sequences coding for variants of CD16. Homologous polynucleotide sequences also include are naturally occurring allelic variations related to SEQ ID NO:1. haNK® cell comprising any polynucleotide encoding a polypeptide having the amino acid sequence shown in either SEQ ID. NO: 1 or SEQ ID NO: 2, a naturally occurring variant thereof, or a sequence that is at least 70% identical, or at least 80%, 90%, or 95% identical to SEQ ID. NO: 1 or SEQ ID NO: 2 is within the scope of the disclosure. In some embodiments, homologous polynucleotide sequences encode conservative amino acid substitutions in SEQ ID. NO: 1 or SEQ ID NO: 2. In some embodiments, haNK® cells generated by transfecting NK-92® cells using a degenerate homologous CD16 polynucleotide sequence that differs from a native polynucleotide sequence but encodes the same polypeptide.

In other examples, cDNA sequences having polymorphisms that change the CD16 amino acid sequences are used to modify the NK-92® cells to produce haNK® cells, such as, for example, the allelic variations among individuals that exhibit genetic polymorphisms in CD16 genes. In other instances, CD16 genes from other species with a polynucleotide sequence that differs from the sequence of SEQ ID NO: 1 are used to modify NK-92® cells.

Variant polypeptides can be made using methods known in the art, such as oligonucleotide-mediated (site-directed) mutagenesis, alanine scanning, and PCR mutagenesis. Site direct mutagenesis cassette mutagenesis, restriction selection mutagenesis, or other known techniques can be performed on the cloned DNA to produce CD16 variants and/or variants to any other polypeptides disclosed herein (for example, a CAR or the antigen-binding portion thereof).

In some embodiments, a polynucleotide encoding a CD16 is mutated to alter the amino acid sequence encoding for CD16 without altering the function of CD16. For example, polynucleotide substitutions leading to amino acid substitutions at “non-essential” amino acid residues can be made in SEQ ID NO:1 or SEQ ID NO:2.

Conservative substitutions in SEQ ID. NO: 1 or SEQ ID NO:2, whereby an amino acid of one class is replaced with another amino acid of the same class, fall within the scope of the disclosed CD16 variants as long as the substitution does not materially alter the activity of the polypeptide. Conservative substitutions are well-known to one of skill in the art. Non-conservative substitutions that affect (1) the structure of the polypeptide backbone, such as a β-sheet or α-helical conformation, (2) the charge, (3) the hydrophobicity, or (4) the bulk of the side chain of the target site can modify CD16 polypeptide function or immunological identity. Non-conservative substitutions entail exchanging a member of one of these classes for another class. Substitutions may be introduced into conservative substitution sites or more preferably into non-conserved sites.

In some embodiments, CD16 polypeptide variants are at least 200 amino acids in length and have at least 70% amino acid sequence identity, or at least 80%, or at least 90% identity to SEQ ID NO:1 or SEQ ID NO:2. In some embodiments, CD16 polypeptide variants are at least 225 amino acid in length and have at least 70% amino acid sequence identity, or at least 80%, or at least 90% identity to SEQ ID NO:1 or SEQ ID NO:2. In some embodiments, CD16 polypeptide variants have a valine at position 158 as determined with reference to SEQ ID NO: 2.

In some embodiments a nucleic acid encoding a CD16 polypeptide may encode a CD16 fusion protein. A CD16 fusion polypeptide includes any portion of CD16 or an entire CD16 fused with a non-CD16 polypeptide. Fusion polypeptides are conveniently created using recombinant methods. For example, a polynucleotide encoding a CD16 polypeptide such as SEQ ID NO:1 or SEQ ID NO:2 is fused in-frame with a non-CD16 encoding polynucleotide (such as a polynucleotide sequence encoding a signal peptide of a heterologous protein). In some embodiment, a fusion polypeptide may be created in which a heterologous polypeptide sequence is fused to the C-terminus of CD16 or is positioned internally in the CD16. Typically, up to about 30% of the CD16 cytoplasmic domain may be replaced. Such modification can enhance expression or enhance cytotoxicity (e.g., ADCC responsiveness). In other examples, chimeric proteins, such as domains from other lymphocyte activating receptors, including but not limited to Ig-a, Ig-B, CD3-e, CD3-d, DAP-12 and DAP-10, replace a portion of the CD16 cytoplasmic domain.

Fusion genes can be synthesized by conventional techniques, including automated DNA synthesizers and PCR amplification using anchor primers that give rise to complementary overhangs between two consecutive gene fragments that can subsequently be annealed and reamplified to generate a chimeric gene sequence (Ausubel, 2002). Many vectors are commercially available that facilitate sub-cloning CD16 in-frame to a fusion moiety.

### Cytokines

The cytotoxicity of NK-92 cells, e.g., the haNK® cells, is dependent on the presence of cytokines (e.g., interleukin-2 (IL-2)). The cost of using exogenously added IL-2 needed to maintain and expand NK-92 cells in commercial scale culture is significant. The administration of IL-2 to human subjects in sufficient quantity to continue activation of NK92 cells would cause adverse side effects.

In one embodiment, haNK-92® cells are modified to express at least one cytokine. In particular, the at least one cytokine is IL-2 (SEQ ID NO:3), IL-12, IL-15, IL-18, IL-21, or a variant thereof. In some embodiments, the cytokine is IL-2 or a variant thereof. In certain embodiments, the IL-2 is a variant that is targeted to the endoplasmic reticulum.

In one embodiment, the IL-2 is cloned and expressed with a signal sequence that directs the IL-2 to the endoplasmic reticulum (erIL-2) (SEQ ID NO: 4). This permits expression of IL-2 at levels sufficient for autocrine activation, but without releasing IL-2 extracellularly. See Konstantinidis et al “Targeting IL-2 to the endoplasmic reticulum confines autocrine growth stimulation to NK-92 cells” Exp Hematol. 2005 February; 33 (2): 159-64. Continuous activation of the FcR-expressing NK-92 cells can be prevented, e.g., by the presence of the suicide gene.

### Suicide Gene

The term “suicide gene” refers to a transgene that allows for the negative selection of cells expressing the suicide gene. A suicide gene is used as a safety system, allowing cells expressing the gene to be killed by introduction of a selective agent. This is desirable in case the recombinant gene causes a mutation leading to uncontrolled cell growth, or the cells themselves are capable of such growth. A number of suicide gene systems have been identified, including the herpes simplex virus thymidine kinase (TK) gene, the cytosine deaminase gene, the varicella-zoster virus thymidine kinase gene, the nitroreductase gene, the Escherichia coli gpt gene, and the E. coli Deo gene. Typically, the suicide gene encodes for a protein that has no ill effect on the cell but, in the presence of a specific compound, will kill the cell. Thus, the suicide gene is typically part of a system.

In one embodiment, the suicide gene is active in haNK® cells. In one embodiment, the suicide gene is the thymidine kinase (TK) gene. The TK gene may be a wild-type or mutant TK gene (e.g., tk30, tk75, sr39tk). Cells expressing the TK protein can be killed using ganciclovir.

In another embodiment, the suicide gene is cytosine deaminase, which is toxic to cells in the presence of 5-fluorocytosine. Garcia-Sanchez et al. “Cytosine deaminase adenoviral vector and 5-fluorocytosine selectively reduce breast cancer cells 1 million-fold when they contaminate hematopoietic cells: a potential purging method for autologous transplantation.” Blood. 1998 Jul. 15; 92 (2): 672-82.

In another embodiment, the suicide gene is cytochrome P450, which is toxic in the presence of ifosfamide or cyclophosphamide. See, e.g. Touati et al. “A suicide gene therapy combining the improvement of cyclophosphamide tumor cytotoxicity and the development of an anti-tumor immune response.” Curr Gene Ther. 2014; 14 (3): 236-46.

In another embodiment, the suicide gene is iCasp9. Di Stasi, (2011) “Inducible apoptosis as a safety switch for adoptive cell therapy.” N Engl J Med 365:1673-1683. See also Morgan, “Live and Let Die: A New Suicide Gene Therapy Moves to the Clinic” Molecular Therapy (2012); 20:11-13. iCasp9 induces apoptosis in the presence of a small molecule, AP1903. AP1903 is biologically inert small molecule, that has been shown in clinical studies to be well tolerated, and has been used in the context of adoptive cell therapy.

### IL-15 Superagonist

For purposes of this disclosure, an IL-15 superagonist refers to an IL-15 receptor complex that can promote NK cell growth and development. In some embodiments, the method of treating chordoma comprises administering an IL-15 superagonist in combination with the NK cell therapy as disclosed above. As disclosed above, in some embodiments, the method comprises administering an IL-15 superagonist to activate the NK cells. One example of IL-15 superagonist is N-803 (formerly ALT-803). N-803 is a clinical grade IL-15 superagonist complex known to induce proliferation and activation of NK and T cell immune compartments (9, 10). N-803 comprises two protein subunits of a human IL-15 variant associated with high affinity to a dimeric human IL-15 receptor α (IL-15Rα) sushi domain/human IgG1 Fc fusion protein. The IL-15 variant is a 114 amino acid polypeptide comprising the mature human IL-15 cytokine sequence, with an asparagine to aspartate substitution at position 72 of helix C (N72D) and exhibits enhanced biological activity (Zhu et al. J. immunol. 2009; 183:3598-607). The human IL-15Rα sushi domain/human IgG1 Fc fusion protein comprises the sushi domain of the human IL-15 receptor α subunit (IL-15Rα) (amino acids 1-65 of the mature human IL-15Rα protein) linked to the human IgG1 CH2-CH3 region containing the Fc domain (232 amino acids). Except for the N72D substitution, all of the protein sequences are human. N-803 is manufactured by Altor Biosciences. ALT-803 and representative sequences are described in U.S. patent publication number 2014/0134128, which is incorporated by reference herein.

Notable anti-tumor effects and augmentation of NK cell-mediated cytotoxicity (11-14) by N-803 in preclinical cancer models has supported several clinical trials focused on N-803 as monotherapy (10), for example, NCT03054909, NCT02099539, NCT01946789) and in combination with other immunotherapeutic agents (15, 16), NCT03853317, NCT03022825, NCT03127098. Thus, in some embodiments, the method for treating chordoma comprises administering both (i) N-601 or the haNK® cells expressing a PD-L1 CAR and (ii) N-803, where N-803 can stimulate anti-tumor NK cell responses in preclinical models of chordoma.

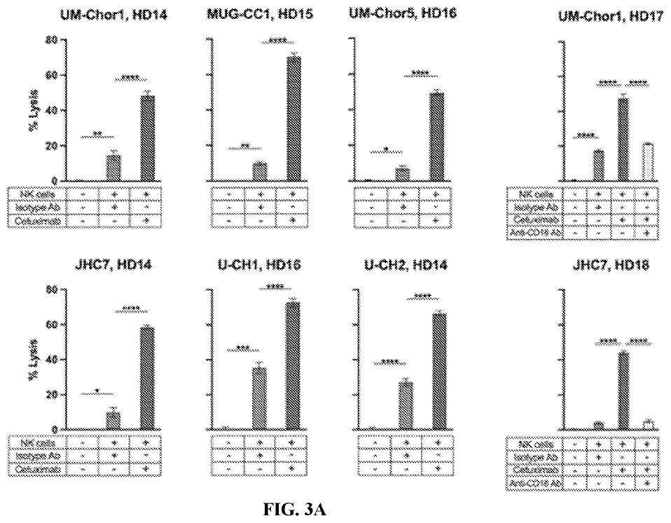

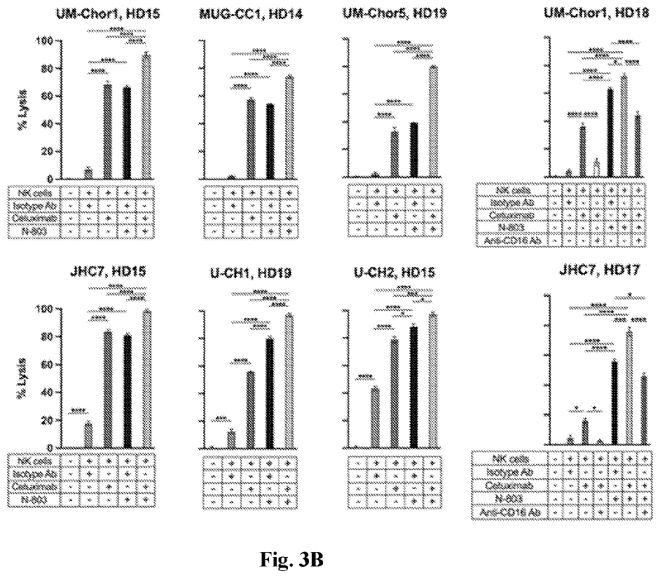

A shown in FIG. 2, the susceptibility of chordoma cell to lysis by healthy donor NK cells was enhanced by N-601 (anti-PD-L1), N-803 (IL-15 superagonist), and combination treatment with N-601 and N-803. This indicates that administering an IL-15 superagonist (e.g., N-803) in combination with the NK cell therapy can boost the efficacy of the NK cell therapy on treating chordoma. The IL-15 superagonist can enhance ADCC-mediated lysis of chordoma cells by the NK cells alone or NK cells in combination of a therapeutic antibody (e.g., anti-EGFR). See FIG. 3B.

### Vectors

Described herein are vectors for transfecting cells to produce the modified cells described herein. In one embodiment, the vectors described herein are transient expression vectors. Exogenous transgenes introduced using such vectors are not integrated in the nuclear genome of the cell; therefore, in the absence of vector replication, the foreign transgenes will be degraded or diluted over time.

In one embodiment, the vectors described herein allow for stable transfection of cells. In one embodiment, the vector allows incorporation of the transgene(s) into the genome of the cell. In one embodiment, the vectors have a positive selection marker. Positive selection markers include any genes that allow the cell to grow under conditions that would kill a cell not expressing the gene. Non-limiting examples include antibiotic resistance, e.g. geneticin (Neo gene from Tn5).

In one embodiment, the vector is a plasmid vector. In one embodiment, the vector is a viral vector. As would be understood by one of skill in the art, any suitable vector can be used. Suitable vectors are well-known in the art.

In some embodiments, the cells are transfected with mRNA encoding the protein of interest (e.g., a CAR). Transfection of mRNA results in transient expression of the protein. In one embodiment, transfection of mRNA into the haNK® cells is performed immediately prior to administration of the cells. In one embodiment, “immediately prior” to administration of the cells refers to between about 15 minutes and about 48 hours prior to administration. Preferably, mRNA transfection is performed about 5 hours to about 24 hours prior to administration.

### Codon Optimization

In some embodiments, the sequence of the constructs used to transform the aNK cells are codon-optimized to maximize expression efficiency of PD-L1 CAR, CD16, and/or erIL-2 in human systems. Codon optimization is typically performed by modifying a nucleic acid sequence by replacing at least one, more than one, or a significant number, of codons in the native sequence with codons that are more frequently or most frequently used in the gene of the expression system. Codon optimization can be used to the rate of translation or to produce recombinant RNA transcripts having desirable properties, such as a longer half-life, as compared with transcripts produced using a non-optimized sequence. Methods for codon optimization are readily available, for example, GeneArt™, from Thermo Fisher Scientific (Waltham, MA); Optimizer, accessible free of charge at http://genomes.urv.es/OPTIMIZER, and GeneGPS® Expression Optimization Technology from DNA 2.0 (Newark, California).

### Transgene Expression

Transgenes can be engineered into an expression vector by any mechanism known to those of skill in the art. Where multiple transgenes are to be inserted into a cell, transgenes may be engineered into the same expression vector or a different expression vector.

In some embodiments, the cells are transfected with mRNA encoding the transgenic protein to be expressed.

Transgenes and mRNA can be introduced into the haNK® cells using any transfection method known in the art, including, by way of non-limiting example, infection, electroporation, lipofection, nucleofection, or “gene-gun.”

Combination with Therapeutic Antibodies and/or Other Anti-Cancer Agents

In some embodiments, haNK® cells or NK cells of the present disclosure are used in combination with therapeutic antibodies and/or other anti-cancer agents. Therapeutic antibodies may be used to target cells that express cancer-associated or tumor-associated markers. Examples of cancer therapeutic monoclonal antibodies are shown in Table 3. In some embodiments, the haNK® cells express an Fc receptor, e.g., a high-affinity Fc receptor that has the sequence set forth in SEQ ID NO:2. In one embodiment, the therapeutic antibody is an anti-EGFR antibody.

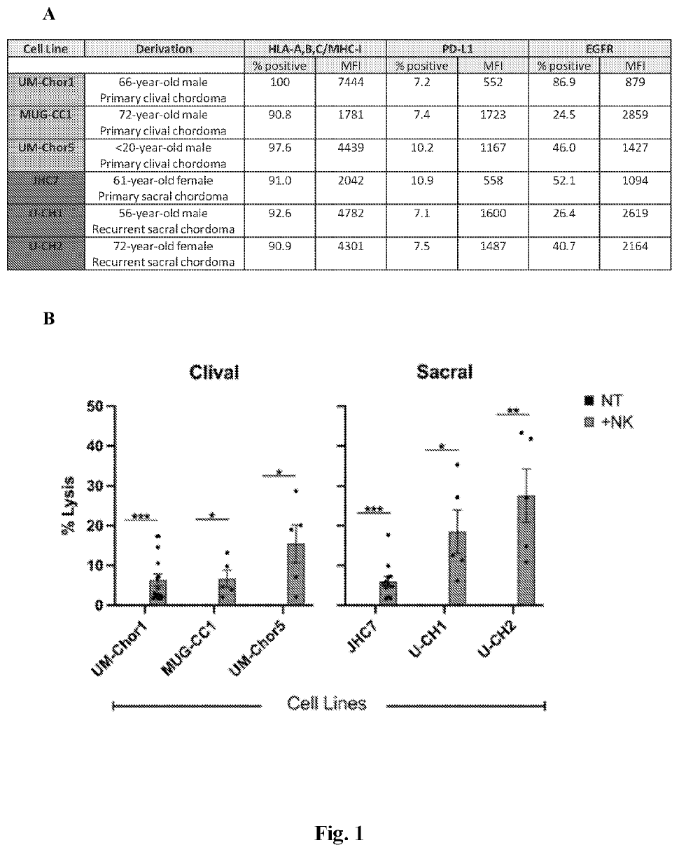

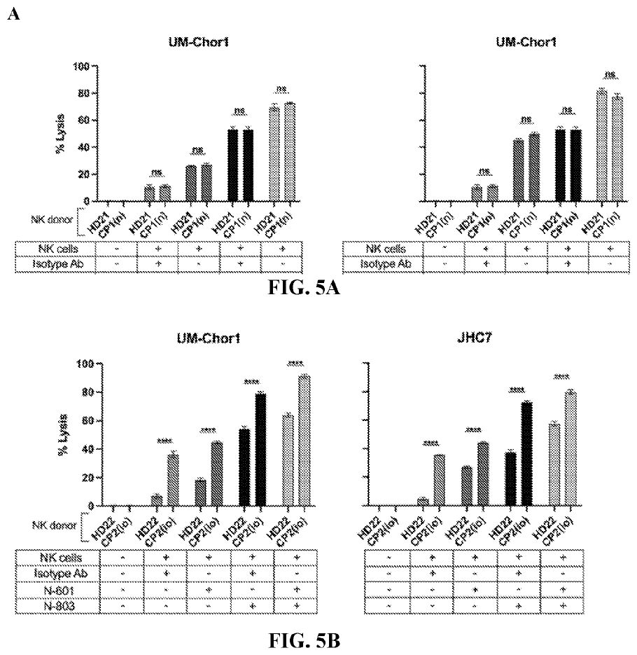

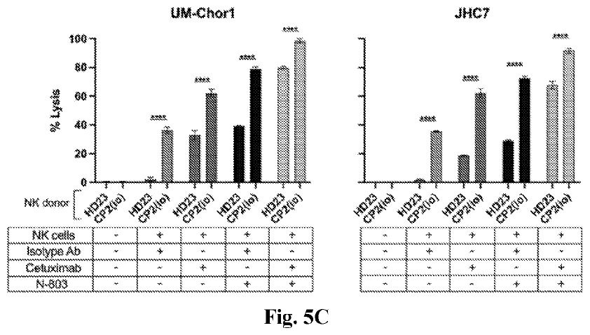

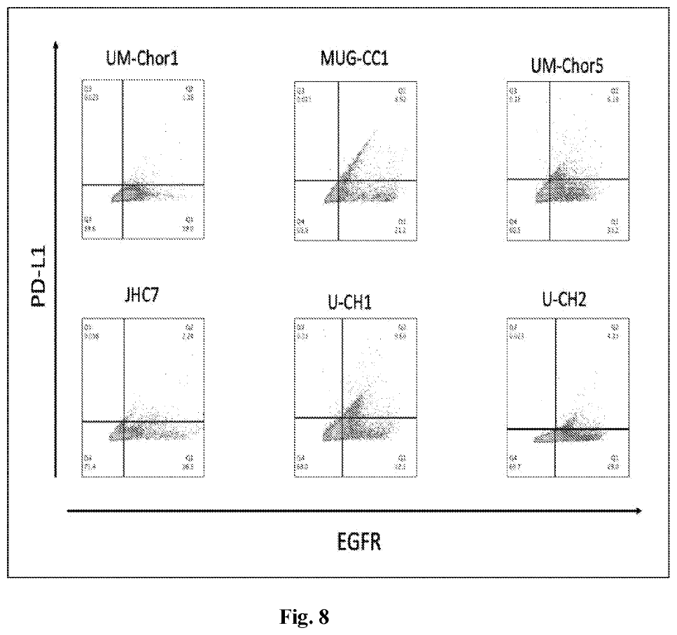

Studies have shown that many chordoma cell lines express high levels of EGFR, and in some cases, the EGFR expression level is higher than that of the PD-L1. See FIG. 1A and FIG. 8. Furthermore, PD-L1-expressing cells also tend to express EGFR, as evidenced by the strong cytotoxicity of cetuximab-mediated ADCC (FIG. 3). The combination of anti-PD-L1 antibody (e.g., N-601), anti EGFR, and an IL-15 superagonist also enhanced NK mediated lysis of chordoma cells. See FIGS. 5A and 5B. Thus in some approaches, methods of treating chordoma comprise administering to a patient having chordoma a plurality of haNK® cells comprising a CAR comprising a means for binding PD-L1 and a CAR comprising a means for binding another tumor-specific antigen (e.g., EGFR). In some approaches, the method of treating chordoma comprises administering to a patient having chordoma a plurality of NK cells and a means for binding PD-L1 and a means for binding another tumor-specific antigen (e.g., EGFR). In some embodiments, the method further comprises administering an IL-15 superagonist to activate the NK cells and further boost the cytotoxicity of the NK cells.

TABLE 3
Illustrative therapeutic monoclonal antibodies
Examples of FDA-approved therapeutic monoclonal antibodies

| | Brand | | | Indication |
|---|---|---|---|---|
| Antibody | name | Company | Target | (Targeted disease) |
| Alemtuzumab | Campath ® | Genzyme | CD52 | Chronic lymphocytic |
| | | | | leukemia |
| Brentuximab | Adcetris ® | | CD30 | Anaplastic large cell |
| vedotin | | | | lymphoma (ALCL) and |
| | | | | Hodgkin lymphoma |
| Cetuximab | Erbitux ® | Bristol-Myers | epidermal growth | Colorectal cancer, Head |
| | | Squibb/Eli | factor receptor | and neck cancer |
| | | Lilly/Merck | | |
| | | KGaA | | |
| Gemtuzumab | Mylotarg ® | Wyeth | CD33 | Acute myelogenous |
| | | | | leukemia (with |
| | | | | calicheamicin) |
| Ibritumomab | Zevalin ® | Spectrum | CD20 | Non-Hodgkin lymphoma |
| tiuxetan | | Pharmaceuticals, | | (with Yttrium-90 or |
| | | Inc. | | indium-111) |
| Ipilimumab | Yervoy ® | | blocks CTLA-4 | Melanoma |
| (MDX-101 ) | | | | |
| Ofatumumab | Arzerra ® | | CD20 | Chronic lymphocytic |
| | | | | leukemia |
| Palivizumab | Synagis ® | MedImmune | an epitope of the RSV | Respiratory Syncytial |
| | | | F protein | Virus |
| Panitumumab | Vectibix ® | Amgen | epidermal growth | Colorectal cancer |
| | | | factor receptor | |
| Rituximab | Rituxan ®, | Biogen | CD20 | Non-Hodgkin lymphoma |
| | Mabthera ® | Idec/Genentech | | |
| Tositumomab | Bexxar ® | GlaxoSmithKline | CD20 | Non-Hodgkin lymphoma |
| Trastuzumab | Herceptin ® | Genentech | ErbB2 | Breast cancer |
| Blinatunomab | | | bispecific CD19- | Philadelphia |
| | | | directed CD3 T-cell | chromosome-negative |
| | | | engager | relapsed or refractory |
| | | | | B cell precursor acute |
| | | | | lymphoblastic |
| | | | | leukemia (ALL) |
| Avelumab | Bavencio ® | Merck KGaA | anti-PD-L1 | Non-small cell lung |
| | | and Pfizer and | | cancer, metastatic |
| | | Eli Lilly | | Merkel cell carcinoma; |
| | | | | gastic cancer, breast |
| | | | | cancer, ovarian cancer, |
| | | | | bladder cancer, |
| | | | | melanoma, |
| | | | | meothelioma, |
| | | | | including metastatic or |
| | | | | locally advanced solid |
| | | | | tumors |
| Daratumumab | | | CD38 | Multiple myeloma |
| Elotuzumab | | | a SLAMF7-directed | Multiple myeloma |
| | | | (also known as CD | |
| | | | 319) | |
| | | | immunostimulatory | |
| | | | antibody | |

Administration of such haNK® cells or NK cells may be carried out simultaneously with the administration of the monoclonal antibody, or in a sequential manner. In some embodiments, the haNK® cells or the NK cells are administered to the subject after the subject has been treated with the monoclonal antibody. Alternatively, the haNK® cells may be administered at the same time, e.g., within 24 hours, of the monoclonal antibody.

In some embodiments, the monoclonal antibody is an anti-EGFR antibody. Suitable anti-EGFR antibodies include clinically approved cetuximab and panitumumab, as well as human and non-human antibodies such as ab52894, ab131498, ab231, ab32562, ab32077, or ab76153 (all commercially available from Abcam, USA), as well as AY13 (Biolegend, USA) and 06-847 (Millipore, USA).

It should be appreciated that additional therapeutic interventions may be used with or complement contemplated treatments. For example, suitable treatments include radiation and/or chemotherapy using agents such as irinotecan, gemcitabine, capecitabine, 5-FU, FOLFIRI, FOLFOX, and/or oxiplatin. In further contemplated aspects, contemplated treatments may also include immune modifiers such as IL15, IL15 agonists, interferon-gamma to increase PD-L1 expression, and/or other checkpoint inhibitors targeting checkpoint receptors and/or their ligands (e.g., PD-1 antibody).

### Therapeutic Applications

In some embodiments, the method to treat chordoma in a subject comprises administering to the subject a therapeutically effective amount of the haNK® cells as described above, thereby treating chordoma. In some embodiments, the haNK® cells express a PD-L1 CAR and a second CAR against another tumor-specific antigen, wherein the second CAR comprises an amino acid sequence having at least 85% identity to a sequence selected from the group consisting of SEQ ID NO: 6-20. In some embodiments, the haNK® cells express an Fc receptor, e.g., a high affinity Fc receptor that has the sequence set forth in SEQ ID NO:2. In some embodiments the method of treating chordoma comprises administering NK cells (e.g., autologous NK cells), a means for binding PD-L1 (e.g., an anti-PD-L1 antibody), and/or a means for binding EGFR (e.g., an anti-EGFR antibody). In some embodiments, the method further comprises administering an IL-15 superagonist, such as N-803.

In further contemplated embodiments, the NK cells or the haNK® cells will be irradiated before transfusion to prevent continuous cell division. While not limiting to the inventive subject matter, the cells will typically be irradiated under conditions that abrogate cell division, but still allow for metabolic activity and cell function such as cytotoxic cell killing. Therefore, suitable radiation dosages will be between 50 cGy and 2,000 cGy. Furthermore, such radiation is typically beta or gamma radiation; however, other manners, such as e-beam irradiation, are also expressly contemplated herein.

The haNK® cells or NK cells can be administered to an individual by absolute numbers of cells, e.g., said individual can be administered from about 1000 cells/injection to up to about 10 billion cells/injection, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) haNK® cells per injection, or any ranges between any two of the numbers, end points inclusive. Therefore, this disclosure also provides a composition comprising a plurality of haNK® cells, wherein the number of cells are 1×10⁸, 1×10⁷, 5×10⁷, 1×10⁶, 5×10⁶, 1×10⁵, 5×10⁵, 1×10⁴, 5×10⁴, 1×10³, or 5×10³ (and so forth).

In other embodiments, said individual can be administered from about 1000 cells/injection/m² to up to about 10 billion cells/injection/m², such as at about, at least about, or at most about, 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m² (and so forth) haNK® cells per injection, or any ranges between any two of the numbers, end points inclusive.

In other embodiments, haNK® cells or NK cells can be administered to such individual by relative numbers of cells, e.g., said individual can be administered about 1000 cells to up to about 10 billion cells per kilogram of the individual, such as at about, at least about, or at most about, 1×10⁸, 1×10⁷, 5×10⁷, 1×10⁶, 5×10⁶, 1×10⁵, 5×10⁵, 1×10⁴, 5×10⁴, 1×10³, or 5×10³ (and so forth) haNK® cells per kilogram of the individual, or any ranges between any two of the numbers, end points inclusive.

In other embodiments, the total dose may be calculated by m² of body surface area, including about 1×10¹¹, 1×10¹⁰, 1×10⁹, 1×10⁸, 1×10⁷, per m², or any ranges between any two of the numbers, end points inclusive. The average person is about 1.6 to about 1.8 m². In a preferred embodiment, between about 1 billion and about 3 billion haNK® cells are administered to a patient. In other embodiments, the amount of haNK® cells injected per dose may be calculated by m² of body surface area, including 1×10¹¹, 1×10¹⁰, 1×10⁹, 1×10⁸, and 1×10⁷, per m². The average body surface area for a person is 1.6-1.8 m².

In other embodiments, haNK® cells or NK cells can be administered to such individual by relative numbers of cells, e.g., said individual can be administered about 1000 cells to up to about 10 billion cells per kilogram of the individual, such as at about, at least about, or at most about, 5×10⁸, 1×10⁸, 5×10⁷, 1×10⁷, 1×10⁶, 5×10⁶, 1×10⁵, 5×10⁵, 1×10⁴, 5×10⁴, 1×10³, or 5×10³ (and so forth) haNK® cells per kilogram of the individual, or any ranges between any two of the numbers, end points inclusive.

haNK® cells or NK cells can be administered once to a patient with cancer, or they can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10 or more weeks during therapy, or any ranges between any two of the numbers, end points inclusive.

In some embodiments, haNK® cells or NK cells are administered in a composition comprising the haNK® cells and a medium, such as human serum or an equivalent thereof. In some embodiments, the medium comprises human serum albumin. In some embodiments, the medium comprises human plasma. In some embodiments, the medium comprises about 1% to about 15% human serum or human serum equivalent. In some embodiments, the medium comprises about 1% to about 10% human serum or human serum equivalent. In some embodiments, the medium comprises about 1% to about 5% human serum or human serum equivalent. In a preferred embodiment, the medium comprises about 2.5% human serum or human serum equivalent. In some embodiments, the serum is human AB serum. In some embodiments, a serum substitute that is acceptable for use in human therapeutics is used instead of human serum. Such serum substitutes may be known in the art, or developed in the future. Although concentrations of human serum over 15% can be used, it is contemplated that concentrations greater than about 5% will be cost-prohibitive. In some embodiments, haNK® cells are administered in a composition comprising haNK® cells and an isotonic liquid solution that supports cell viability. In some embodiments, haNK® cells are administered in a composition that has been reconstituted from a cryopreserved sample.

Pharmaceutically acceptable compositions comprising the haNK® cells or NK cells can include a variety of carriers and excipients. A variety of aqueous carriers can be used, e.g., buffered saline and the like. These solutions are sterile and generally free of undesirable matter. Suitable carriers and excipients and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject. As used herein, the term pharmaceutically acceptable is used synonymously with physiologically acceptable and pharmacologically acceptable. A pharmaceutical composition will generally comprise agents for buffering and preservation in storage and can include buffers and carriers for appropriate delivery, depending on the route of administration.

These compositions for use in in vivo or in vitro may be sterilized by sterilization techniques employed for cells. The compositions may contain acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cells in these formulations and/or other agents can vary and will be selected primarily based on fluid volumes, viscosities, body weight and the like in accordance with the particular mode of administration selected and the subject's needs.

In one embodiment, haNK® cells or NK cells are administered to the patient in conjunction with one or more other treatments or agent as disclosed above. In some embodiments, the one or more other treatments for the cancer being treated include, for example, an antibody, radiation, chemotherapeutic, stem cell transplantation, or hormone therapy.

In some embodiments, haNK® cells or the NK cells and the other cancer agent/treatment are administered simultaneously or approximately simultaneously (e.g., within about 1, 5, 10, 15, 20, or 30 minutes of each other). In some embodiments, the haNK® cells and the other cancer agent/treatment are administered sequentially. In some embodiments, the other cancer treatment/agent is administered one, two, or three days after the administration of the haNK® cells.

In one embodiment, the other cancer agent is an antibody. In one embodiment, haNK® cells are administered in conjunction with an antibody targeting the diseased cells (e.g., an anti-EGFR antibody). In some embodiments, the administration of the antibody and the haNK® cells is contemporaneous such that both the antibody and the haNK® cells are present in the patient's blood in measurable quantities at the same time. Consequently, co-administration of the antibody and the haNK® cells may be performed at the same time, or within 10 minutes or within 30 minutes or within 2 hours of each other. In one embodiment, haNK® cells and an antibody are administered to the patient in the same formulation (and administered to the patient at the same time). In one embodiment, haNK® cells and the antibody are administered in separate formulations and are administered concurrently or separately (e.g., on different dosing schedules or at different times of the day). Suitable dosages for administration of the antibody will typically be between 100 mg/m² and 1,000 mg/m², or between 100 mg/m² and 300 mg/m², or between 300 mg/m² and 600 mg/m², or between 600 mg/m² and 900 mg/m², or even higher. The antibody can be administered via any suitable route, such as intravenous or intra-tumoral. In some embodiments, the antibody is administered intravenously over a period of between about 1 min and 120 min, and more typically between about 10 min and 60 min.

### Kits

Also disclosed are kits for the treatment of chordoma using compositions comprising a plurality of haNK® cells or NK cells as described herein. In some embodiments, the kits of the present disclosure comprises a plurality of of haNK® cells, each comprising a first chimeric antigen receptor (CAR) comprising a means for binding PD-L1 and a second chimeric antigen receptor comprising an amino acid sequence having at least 85% identity to a sequence selected from the group consisting of SEQ ID NOs: 6-20. In some embodiments, the kit also includes at least one monoclonal antibody against a tumor-specific antigen as disclosed herein. In some embodiments, the haNK® cell further expresses an IL-2, e.g., an erIL-2 and express an Fc receptor.

Also disclosed herein is a kit for treating chordoma in a subject in need thereof. In some embodiments, the kit comprises (i) NK cells and (ii) means for binding PD-L1 and/or a means for binding EGFR. In some embodiments, the means for binding PD-L1 or the means for binding EGFR are both monoclonal antibodies. In some embodiments, the kit comprises an an IL-15 superagonist, such as N-803.

In certain embodiments, the kit may contain additional compounds such as therapeutically active compounds or drugs that are to be administered before, at the same time or after administration of haNK® cells. Examples of such compounds include an antibody, vitamins, minerals, fludrocortisone, ibuprofen, lidocaine, quinidine, chemotherapeutic, and the like.

In various embodiments, instructions for use of the kits will include directions to use the kit components in the treatment of a cancer or an infectious disease. The instructions may further contain information regarding how to handle the haNK® cells (e.g., thawing and/or culturing). The instructions may further include guidance regarding the dosage and frequency of administration.

In certain embodiments, the kit further comprises one or more containers filled with one or more compositions described herein, e.g., a composition comprising haNK® cells as described herein. Optionally associated with such containers can be a label indicating the kit is for treating a cancer, such as those described herein. Optionally the label also includes a notice in the form prescribed by a governmental agency regulating the manufacture, use or sale of pharmaceuticals or biological products, which notice reflects approval by the agency of manufacture, use or sale for human administration.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, and the like. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### REFERENCES

1. C. R. Heery, Chordoma: The Quest for Better Treatment Options. Oncol Ther 4, 35-51 (2016).
2. Chugh et al., Chordoma: the nonsarcoma primary bone tumor. Oncologist 12, 1344-1350 (2007).
3. S. Stacchiotti, J. Sommer, G. Chordoma Global Consensus, Building a global consensus approach to chordoma: a position paper from the medical and patient community. Lancet Oncol 16, e71-83 (2015).
4. C. M. McPherson et al., Metastatic disease from spinal chordoma: a 10-year experience. J Neurosurg Spine 5, 277-280 (2006).
5. Y. Pan, L. Lu, J. Chen, Y. Zhong, Z. Dai, Analysis of prognostic factors for survival in patients with primary spinal chordoma using the SEER Registry from 1973 to 2014. J Orthop Surg Res 13, 76 (2018).
6. C. R. Heery et al., Phase I Trial of a Yeast-Based Therapeutic Cancer Vaccine (GI-6301) Targeting the Transcription Factor Brachyury. Cancer Immunol Res 3, 1248-1256 (2015).
7. P. J. DeMaria et al., Randomized, Double-Blind, Placebo-Controlled Phase II Study of Yeast-Brachyury Vaccine (GI-6301) in Combination with Standard-of-Care Radiotherapy in Locally Advanced, Unresectable Chordoma. Oncologist 26, e847-e858 (2021).
8. R. Fujii et al., Enhanced killing of chordoma cells by antibody-dependent cell-mediated cytotoxicity employing the novel anti-PD-L1 antibody avelumab. Oncotarget 7, 33498-33511 (2016).
9. K. M. Knudson, K. C. Hicks, S. Alter, J. Schlom, S. R. Gameiro, Mechanisms involved in IL-15 agonist enhancement of anti-PD-L1 therapy. J Immunother Cancer 7, 82 (2019).
10. R. Romee et al., First-in-human phase 1 clinical study of the IL-15 agonist complex ALT-803 to treat relapse after transplantation. Blood 131, 2515-2527 (2018).
11. M. Fantini et al., An IL-15 Agonist, ALT-803, Enhances Antibody-Dependent Cell-Mediated Cytotoxicity Elicited by the Monoclonal Antibody NEO-201 Against Human Carcinoma Cells. Cancer Biother Radiopharm 34, 147-159 (2019).
12. M. Rosario et al., The IL-15-Based ALT-803 Complex Enhances FcgammaRIIIa-Triggered NK Cell Responses and In Vivo Clearance of B Cell Lymphomas. Clin Cancer Res 22, 596-608 (2016).
13. B. Liu et al., Evaluation of the biological activities of the IL-15 agonist complex, ALT-803, following intravenous versus subcutaneous administration in murine models. Cytokine 107, 105-112 (2018).
14. C. Jochems et al., Analyses of functions of an anti-PD-L1/TGFbetaR2 bispecific fusion protein (M7824). Oncotarget 8, 75217-75231 (2017).
15. J. M. Wrangle et al., ALT-803, an IL-15 superagonist, in combination with nivolumab in patients with metastatic non-small cell lung cancer: a non-randomised, open-label, phase 1b trial. Lancet Oncol 19, 694-704 (2018).
16. K. Margolin et al., Phase I Trial of ALT-803, A Novel Recombinant IL15 Complex, in Patients with Advanced Solid Tumors. Clin Cancer Res 24, 5552-5561 (2018).
17. L. Walcher et al., Cancer Stem Cells-Origins and Biomarkers: Perspectives for Targeted Personalized Therapies. Front Immunol 11, 1280 (2020).
18. H.-M. Zhou, J.-G. Zhang, X. Zhang, Q. Li, Targeting cancer stem cells for reversing therapy resistance: mechanism, signaling, and prospective agents. Signal Transduction and Targeted Therapy 6, 62 (2021).
19. Z. Yu, T. G. Pestell, M. P. Lisanti, R. G. Pestell, Cancer stem cells. Int J Biochem Cell Biol 44, 2144-2151 (2012).
20. K. P. Fabian et al., PD-L1 targeting high-affinity NK (t-haNK) cells induce direct antitumor effects and target suppressive MDSC populations. J Immunother Cancer 8, (2020).
21. P. Dalerba et al., CDX2 as a Prognostic Biomarker in Stage II and Stage III Colon Cancer. N Engl J Med 374, 211-222 (2016).
22. E. Aydemir et al., Characterization of cancer stem-like cells in chordoma. J Neurosurg 116, 810-820 (2012).
23. M. Safari, A. Khoshnevisan, An overview of the role of cancer stem cells in spine tumors with a special focus on chordoma. World J Stem Cells 6, 53-64 (2014).
24. A. J. Oliver et al., Tissue-Dependent Tumor Microenvironments and Their Impact on Immunotherapy Responses. Front Immunol 9, 70 (2018).
25. K. J. Chambers et al., Incidence and survival patterns of cranial chordoma in the United States. Laryngoscope 124, 1097-1102 (2014).
26. W. Hsu et al., Generation of chordoma cell line JHC7 and the identification of Brachyury as a novel molecular target. J Neurosurg 115, 760-769 (2011).
27. J. H. Owen et al., UM-Chor1: establishment and characterization of the first validated clival chordoma cell line. J Neurosurg 128, 701-709 (2018).
28. S. Bruderlein et al., Molecular characterization of putative chordoma cell lines. Sarcoma 2010, 630129 (2010).
29. S. Scheil et al., Genome-wide analysis of sixteen chordomas by comparative genomic hybridization and cytogenetics of the first human chordoma cell line, U-CH1. Genes Chromosomes Cancer 32, 203-211 (2001).
30. V. Gellner et al., Establishment of clival chordoma cell line MUG-CC1 and lymphoblastoid cells as a model for potential new treatment strategies. Sci Rep 6, 24195 (2016).
31. D. Mathios et al., Therapeutic administration of IL-15 superagonist complex ALT-803 leads to long-term survival and durable antitumor immune response in a murine glioblastoma model. Int J Cancer 138, 187-194 (2016).
32. Y. Feng et al., Expression of programmed cell death ligand 1 (PD-L1) and prevalence of tumor-infiltrating lymphocytes (TILs) in chordoma. Oncotarget 6, 11139-11149 (2015).
33. M. X. Zou et al., Expression of programmed death-1 ligand (PD-L1) in tumor-infiltrating lymphocytes is associated with favorable spinal chordoma prognosis. Am J Transl Res 8, 3274-3287 (2016).
34. G. Scognamiglio et al., Patient-derived organoids as a potential model to predict response to PD-1/PD-L1 checkpoint inhibitors. Br J Cancer 121, 979-982 (2019).
35. I. M. Siu et al., Erlotinib inhibits growth of a patient-derived chordoma xenograft. PLoS One 8, e78895 (2013).
36. B. Dewaele et al., Frequent activation of EGFR in advanced chordomas. Clin Sarcoma Res 1, 4 (2011).
37. A. Shalaby et al., The role of epidermal growth factor receptor in chordoma pathogenesis: a potential therapeutic target. J Pathol 223, 336-346 (2011).
38. B. Boyerinas et al., Antibody-Dependent Cellular Cytotoxicity Activity of a Novel Anti-PD-L1 Antibody Avelumab (MSB0010718C) on Human Tumor Cells. Cancer Immunol Res 3, 1148-1157 (2015).
39. R. Fujii, J. Schlom, J. W. Hodge, A potential therapy for chordoma via antibody-dependent cell-mediated cytotoxicity employing NK or high-affinity NK cells in combination with cetuximab. J Neurosurg 128, 1419-1427 (2018).
40. A. P. de Souza, C. Bonorino, Tumor immunosuppressive environment: effects on tumor-specific and nontumor antigen immune responses. Expert Rev Anticancer Ther 9, 13171332 (2009).
41. K. Rezvani, R. Rouce, E. Liu, E. Shpall, Engineering Natural Killer Cells for Cancer Immunotherapy. Mol Ther 25, 1769-1781 (2017).
42. C. Zhang et al., Chimeric Antigen Receptor-Engineered NK-92 Cells: An Off-the-Shelf Cellular Therapeutic for Targeted Elimination of Cancer Cells and Induction of Protective Antitumor Immunity. Front Immunol 8, 533 (2017).
43. A. Fabian, M. Barok, G. Vereb, J. Szöllösi, Die hard: Are cancer stem cells the Bruce Willises of tumor biology? Cytometry Part A 75A, 67-74 (2009).
44. P. S. Kim et al., IL-15 superagonist/IL-15RalphaSushi-Fc fusion complex (IL-15SA/IL-15RalphaSu-Fc; ALT-803) markedly enhances specific subpopulations of NK and memory CD8+ T cells, and mediates potent anti-tumor activity against murine breast and colon carcinomas. Oncotarget 7, 16130-16145 (2016).
45. G. Lanzino, A. S. Dumont, M. B. Lopes, E. R. Laws, Jr., Skull base chordomas: overview of disease, management options, and outcome. Neurosurg Focus 10, E12 (2001).
46. T. A. Kato et al., In vitro characterization of cells derived from chordoma cell line U-CH1 following treatment with X-rays, heavy ions and chemotherapeutic drugs. Radiation Oncology 6, 116 (2011).
47. C. Maccalli, A. Volontè, C. Cimminiello, G. Parmiani, Immunology of cancer stem cells in solid tumours. A review. European Journal of Cancer 50, 649-655 (2014).
48. R. Castriconi et al., NK Cells Recognize and Kill Human Glioblastoma Cells with Stem Cell-Like Properties. The Journal of Immunology 182, 3530 (2009).
49. E. Ames et al., NK Cells Preferentially Target Tumor Cells with a Cancer Stem Cell Phenotype. The Journal of Immunology 195, 4010 (2015).
50. K. P. Fabian et al., PD-L1 targeting high-affinity NK (t-haNK) cells induce direct antitumor effects and target suppressive MDSC populations. J Immunother Cancer 8, e000450 (2020).
51. Oelsner S, Friede M, Zhang C et al. Continuously expanding CAR NK-92 cells display selective cytotoxicity against B-cell leukemia and lymphoma. Cytotherapy, 2017; 19:235-249.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### EXAMPLES

The following examples are for illustrative purposes only and should not be interpreted as limitations. There are a variety of alternative techniques and procedures available to those of skill in the art which would similarly permit one to successfully perform the examples below.

### Example 1. Materials Used in the Examples

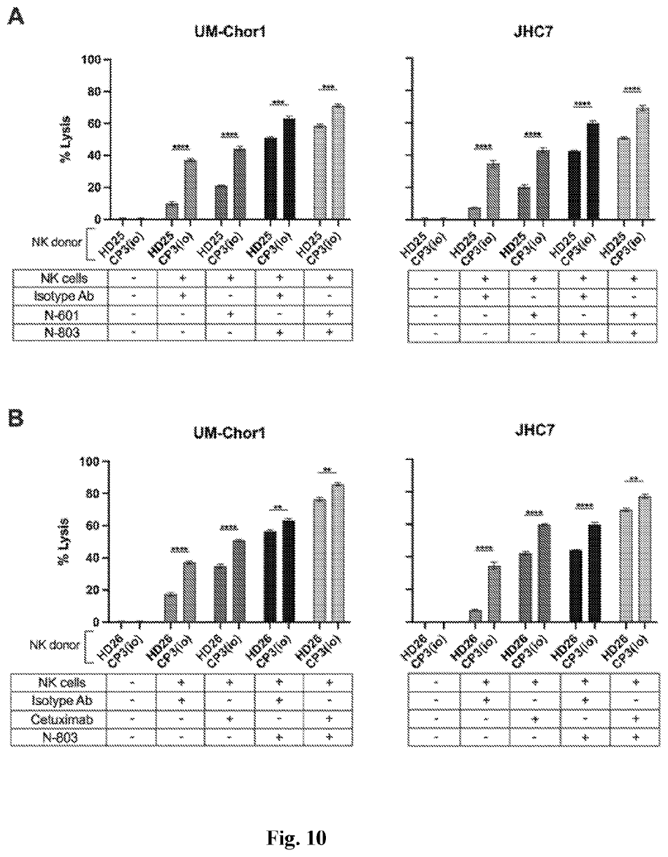

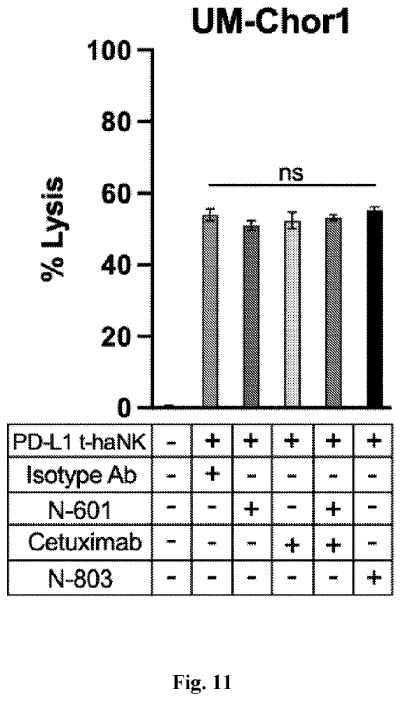

Cell lines: UM-Chor1 (clival), JHC7 (sacral), and U-CH1 (sacral) were kindly provided by the Chordoma Foundation (Durham, NC). U-CH2 (sacral), UM-Chor5 (clival), and MUG-CC1 (clival) were obtained from American Type Culture Collection. All cell lines were passaged twice per week, used at a low (<30) passage number, and serially verified to be free of mycoplasma infection.
Protocols: Healthy donor PBMCs were obtained from the NIH Clinical Center Blood Bank (NCT00001846). Whole blood from a treatment-naïve chordoma patient was collected through a tissue procurement protocol (NCT03429036) between the NIH and Johns Hopkins University Hospital. Whole blood from chordoma patients receiving immuno-oncology therapy (NCT02383498, NCT03173950, NCT03349983, NCT01519817, NCT01772004, NCT02179515, NCT02536469) was obtained from collaborators at the National Institutes of Health. Informed consent was obtained for enrollment on the respective IRB protocol.
Immune reagents and NK cells: An anti-PD-L1 antibody (N-601), an IL-15/IL-15r agonist (N-803), and the PD-L1-specific chimeric antigen receptor engineered high affinity natural killer cells (PD-L1 t-haNKs) were provided by ImmunityBio under a Cooperative Research and Development Agreement (CRADA) with the National Cancer Institute (NCI) of the National Institutes of Health (NIH). Cetuximab (Erbitux®) was obtained from Lilly. Human IgG1 isotype control antibody and human anti-CD16 antibody were obtained from BioLegend.
PBMCs: PBMCs were isolated from chordoma patient whole blood using BD Bioscience Vacutainer CPT tubes according to the manufacturer's protocol. NK cells were isolated from PBMCs using the Miltenyi Biotec Human NK Cell Isolation (negative selection) Kit according to the manufacturer's protocol with >90% purity. NK cells were rested overnight in RPMI 1640 media before being used as effector cells in assays.
Treatment: For experiments using chordoma patient NK cells as effector cells, treatments undergone by patients prior to providing blood for this study are as follows: One patient, CP1(n), had a newly diagnosed clival chordoma and provided blood on the day of primary surgical resection. This patient had not received any other therapies (i.e. radiation, immunotherapy) prior to providing blood (FIG. 5A). A second patient, CP2(io), had a recurrent sacral chordoma and was previously treated with surgical resection, radiation, yeast brachyury vaccine (NCT02383498), anti-PD-1 mAb (NCT03173950), and modified vaccinia Ankara-Fowlpox brachyury vaccine (NCT03349983) (FIG. 5B, C). A third patient, CP3(io), had a recurrent thoracic spine chordoma and was previously treated with surgical resection, radiation, yeast brachyury vaccine (NCT01519817), anti-PD-L1 mAb (NCT01772004), modified vaccinia Ankara-based vaccine modified to express brachyury and T cell co-stimulatory signals (NCT02179515), anti-IL-8 mAb (NCT02536469), and modified vaccinia Ankara-Fowlpox brachyury vaccine (NCT03349983) (FIG. 10).
Flow cytometric analysis and FACS: For general cell surface marker analysis, chordoma cells were labeled with LIVE/DEAD 488 Fixable Green Dead Cell Stain Kit (Life Technologies #L34970) according to the manufacturer's protocol. Cells were then washed and resuspended in FACS buffer (PBS+1% BSA). Human Fc block (BD #564219) was added to 1×10⁶ cells and incubated for 10 minutes on ice. Cells were stained with HLA-A,B,C-APC (BioLegend #311410), PD-L1-BV421 (BD #563738), or EGFR-PE (BD #555997) and fixed with Cytofix (BD #554655). Marker expression was quantified by percent positive cells and mean fluorescence intensity (MFI). Flow cytometry was performed on BD FACSCanto (BD Biosciences) and analyzed using FlowJo v10.7.1 (TreeStar).

The CSC population in the UM-Chor1 cell line was identified as CD24⁺CD15⁺CD133⁺ cells using the following antibodies from BD Biosciences: CD24-BV711 (ML5), CD15-PE (6D4), and CD133-APC (W6D3). To stain for surface markers, PD-L1-BV605 (MPC11, BioLegend), MICA/B-PECy7 (6D4, BioLegend), HLA-A,B,C-BV605 (W6/32, BioLegend) and B7-H6-AlexaFluor700 (875001, R&D) antibodies and their appropriate isotype controls were used. Flow cytometry was performed on BD LSRFortessa (BD Biosciences) and analyzed using FlowJo V.10.7.1 (TreeStar).
¹¹¹Indium NK cell killing assay: NK cells (effector cells) were isolated from healthy donor PBMCs or chordoma patient PBMCs as previously described. NK cells isolated from PBMCs were cultured overnight in the NK cell media described above with or without 50 ng/ml of N-803. All NK cells were washed in the above-described media after overnight incubation. Cryopreserved PD-L1 t-haNK® cells were thawed on the day of the experiment and washed two times in the previously described NK cell media before being adjusted to the desired concentration and plated.

Chordoma cells (target cells) were labeled with ¹¹¹In (10 μL/100,000 cells). NK cells (healthy donor, chordoma patient, or PD-L1 t-haNKs) were added to wells at various effector-to-target (E:T) ratios, depending on the experiment. Specific E:T ratios are indicated in figure legends. After 20 hours, assay plates were centrifuged at 1500 RPM and supernatants were quantified for the presence of ¹¹¹In using a PerkinElmer (Waltham, MA) WIZARD2 Automatic Gamma Counter. Spontaneous ¹¹¹In release was determined by incubating target cells without effector cells, and complete lysis was determined by incubating target cells with 0.05% Triton X-100. Experimental lysis was standardized using the following equation: Percent lysis=[(experimental cpm−spontaneous cpm)/(complete cpm−spontaneous cpm)]×100. Negative control values in each bar graph represent spontaneous lysis of target cells without effector cells. All experiments were carried out in technical triplicate, with each individual experiment being repeated at least three separate times unless otherwise specified.
Flow-based NK cell killing assay: UM-Chor1 cells were labeled with CellTrace Violet Proliferation dye (Thermo Fisher, Waltham, MA) and co-cultured for 4 hours with healthy donor-derived NK cells that had been treated with N-803 overnight. Afterwards, the co-cultures were stained with Live/Dead Fixable Aqua stain (Thermo Fisher) and the CSC marker antibodies described above. Flow cytometry was performed on BD LSRFortessa (BD Biosciences) and analyzed using FlowJo V.10.7.1 (TreeStar). The CSC and non-CSC populations were downsampled using FlowJo such that each cellular subtype would have similar numbers of cells in the histogram.
Statistics: Experiments were carried out in technical triplicate with each experiment being repeated at least three times unless otherwise specified due to limitation by NK cell donor. Comparisons in NK cell killing models of two independent groups were performed using a Student's/test with a 2-tailed distribution at a confidence level of 95%. Comparison of more than two independent groups were performed using one-way analysis of variance (ANOVA) with Tukey's multiple comparisons test. All error bars indicate standard error of the mean (SEM). Test results are reported as P values with a significance cutoff set at P<0.05. All analyses were performed using GraphPad 9.0f software (GraphPad Software Inc., San Diego, CA).

### Example 2. Chordoma Cell Lines from Distinct Anatomic Locations are Susceptible to Lysis by Healthy Donor NK Cells

Preclinical and clinical evidence indicate that tumor immune microenvironments are shaped by tissue of origin and anatomic location, thus impacting disease progression and response to immunotherapies (24). Given the anatomically distinct locations along the axial skeleton where chordoma arises and previously reported differences in survival between cranial chordomas and other locations (25), we first sought to determine whether there is a difference in target surface marker expression between three chordoma cell lines derived from clival chordomas (UM-Chor1, MUG-CC1, UM-Chor5) and three lines from sacral chordomas (JHC7, U-CH1, U-CH2) (26-30). Chordoma tissue samples and cell lines have been shown to express PD-L1 (8, 31-34) and EGFR (35-37), both of which are surface antigens with clinically available monoclonal antibodies that mediate ADCC with NK cells (38, 39). All six chordoma cell lines examined expressed HLA-A,B,C/MHC-1, PD-L1, and EGFR as assessed by % positive cells and mean fluorescent intensity (MFI) (FIG. 1A). There were no statistically significant differences between clival and sacral chordoma cell lines in expression of HLA-A,B,C (% Positivity P=0.18, MFI P=0.67), PD-L1 (% Positivity P=0.89, MFI P=0.89), or EGFR (% Positivity P=0.55, MFI P=0.77).

Next, we interrogated the susceptibility of chordoma cell lines to lysis by healthy donor NK cells and determined whether the site of anatomic derivation had any significant impact on this susceptibility. Consistent with prior studies using in vitro NK cell killing assays (8, 39), the average NK cell-mediated lysis of chordoma cell lines was significantly higher than spontaneous lysis controls but remained low overall (avg. 10.7%, SEM 1.5%), highlighting the potential for augmentation in NK cell-mediated lysis of chordoma cells (FIG. 1B). There was no significant difference between NK cell-mediated lysis of clival and sacral chordoma cell lines (P=0.09), indicating that anatomic derivation of chordoma cell lines does not alter sensitivity to lysis by NK cells.

### Example 3. Anti-PD-L1 Antibody (N-601) Enhances Killing of Chordoma Cells Through ADCC

Our group has demonstrated that a clinically relevant anti-PD-L1 antibody, avelumab, enhances NK cell-mediated lysis of chordoma cells via ADCC (8). We used a recently developed anti-PD-L1 antibody, N-601, to perform similar in vitro ADCC killing assays with healthy donor NK cells as effector cells and chordoma cell lines as target cells. N-601 significantly increased NK cell lysis of 6/6 chordoma cell lines compared to isotype control antibody (FIG. 2A). The average increase in NK cell-mediated tumor cell lysis by N-601 in >20 independent experiments was 6.2-fold (P:≤0.0001) compared to isotype control antibody.

Given that N-601 is a structural homolog of avelumab and maintains the human Fc element, we hypothesized ADCC to be the primary mechanism through which N-601 enhanced NK cell-mediated killing of chordoma cells. Blocking CD16 on NK cells with a neutralizing antibody before co-culture with chordoma cells entirely abrogated NK cell killing of tumor cells (P≤0.0001) (FIGS. 2, A and B), indicating that N-601 enhanced NK cell lysis of chordoma cells through an ADCC-dependent mechanism.

### Example 4. IL-15/IL-15r Agonist (N-803) Enhances NK Cell Killing of Chordoma Cells, and Combinatorial Treatment with Anti-PD-L1 Antibody (N-601) and N-803 Further Enhances Cytotoxicity

N-803 is a humanized IL-15 superagonist that stimulates and expands NK cells and T cells (9, 10). While this immunostimulatory complex has demonstrated antitumor efficacy as a monotherapy and in combination against multiple cancer types in clinical studies NCT03054909, NCT02099539, NCT01946789, NCT03853317, NCT03022825, NCT03127098 (10, 15), it has yet to be explored in chordoma. NK cells treated with N-803 were significantly more cytolytic against chordoma cell lines compared to untreated NK cells (FIG. 2B). The average increase in tumor cell lysis by N-803 treatment of NK cells in >20 independent experiments was 13.6-fold (P≤0.0001) compared to untreated controls.

We hypothesized that combination treatment of chordoma cells with N-601 and NK cells with N-803 would further enhance tumor cell lysis by NK cells compared to monotherapy treatments. This combinatorial approach, which has not yet been evaluated in the literature, resulted in additional cytotoxic effect on chordoma cells when compared to untreated controls (FIG. 2B). The average increase in tumor cell lysis by combinatory treatment with N-601 and N803 in >10 independent experiments was 15.5-fold (P≤0.0001) compared to untreated controls. Combination treatment with N-601 and N-803 significantly enhanced NK cell killing of 6/6 chordoma cell lines compared to monotherapy with N-601 (P≤0.0001) or N-803 (P≤0.05) (FIG. 2B).

To evaluate the contribution of ADCC to NK cell-mediated killing of chordoma cells when N-601 and N-803 are combined, NK cells were treated with a CD16 neutralizing antibody before co-culture with chordoma cells. This resulted in attenuated killing when compared to both combination treatment (P≤0.0001) and N-803 alone (UM-Chor1 P≤0.0001; JHC7 P=0.0056) (FIG. 2B). This result suggests that in the presence of N-601, ADCC still plays a critical role in the lysis of chordoma cells by N-803-activated NK cells.

### Example 5. Anti-EGFR Antibody (Cetuximab) Enhances Killing of Chordoma Cells Through ADCC and Combinatorial Treatment with Cetuximab and IL-15/IL-15r Agonist (N-803) Further Enhances Cytotoxicity

Previous work by our group has also demonstrated that an anti-EGFR mAb, cetuximab, mediates NK cell lysis of chordoma cells via ADCC (39). These findings were confirmed and extended here with additional clival and sacral chordoma cell lines, as we showed that cetuximab increased NK cell lysis of six chordoma cell lines compared to isotype control antibody (FIG. 3A). The average increase in tumor cell lysis by cetuximab in >20 independent experiments was 9.3-fold (P≤0.0001) compared to isotype control antibody. Furthermore, neutralization of CD16 on NK cells with a blocking antibody resulted in abrogation of tumor cell lysis (P≤0.0001) (FIGS. 3, A and B) which is consistent with previous findings that cetuximab enhances NK cell lysis of chordoma cells via ADCC (39).

Given that N-803 enhanced ADCC-mediated lysis of chordoma cells in the presence of N-601, we hypothesized that combinatory treatment with cetuximab and N-803 would similarly result in greater tumor cell lysis than monotherapy approaches. This therapeutic combination, not yet studied in chordoma, resulted in additional cytotoxic effect against chordoma lines when compared to untreated controls as follows (FIG. 3B). The average increase in tumor cell lysis by combinatory treatment with cetuximab and N-803 in >10 independent experiments was 24.2-fold (P≤0.0001) compared to untreated controls. Combination treatment with cetuximab and N-803 significantly enhanced NK cell killing of 6/6 chordoma cell lines compared to cetuximab monotherapy (P≤0.001) and N-803 monotherapy (P≤0.05).

CD16 neutralization on NK cells attenuated killing when compared to combination treatment (P≤0.0001) and monotherapy with N-803 (UM-Chor1 P≤0.0001; JHC7 P=0.01) (FIG. 3B), indicating that cetuximab-mediated ADCC remains essential for increased tumor cell lysis when cetuximab and N-803 are combined.

### Example 6. Doublet Treatment of Chordoma Cells with Anti-PD-L1 (N-601) and Anti-EGFR (Cetuximab) Antibodies Further Enhances Sensitivity to Lysis by IL-15 Superagonist (N-803)-Activated NK Cells

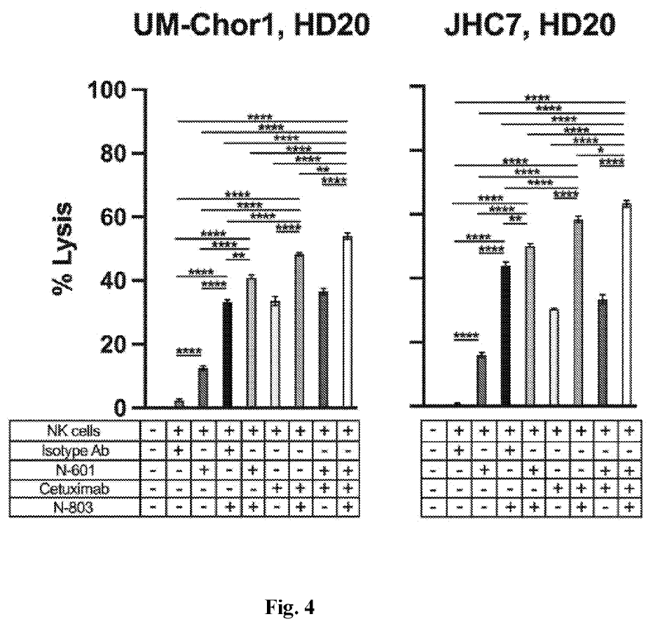

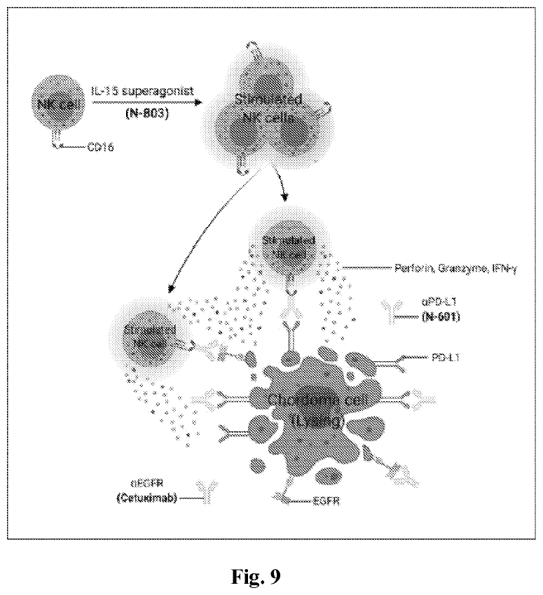

Flow cytometric analysis of six chordoma cell lines revealed higher EGFR expression than PD-L1 expression (FIG. 1A and FIG. 8). Furthermore, PD-L1+ cells also tended to be EGFR+, as evidenced by the robust cytotoxicity of cetuximab-mediated ADCC (FIG. 3). However, we identified small subpopulations of cells that appeared to be PD-L1+/EGFR− (FIG. 8), thus theoretically posing additional anti-tumor activity by combining therapeutic antibodies targeting both surface antigens. To model this doublet ADCC-promoting antibody treatment strategy with N-803-ehanced effector cells (FIG. 9), two chordoma cell lines were treated with N-601 and cetuximab before being targeted by N-803-activated NK cells. This combination approach proved to be marginally more effective than the next best therapeutic combination of cetuximab and N-803 (UM-Chor1 P=0.003; JHC7 P=0.03) (FIG. 4).

### Example 7. Chordoma Patients' NK Cells Mediate Significant Lysis of Chordoma Cells and are Enhanced with Anti-PD-L1 Antibody (N-601), Anti-EGFR Antibody (Cetuximab), and IL-15/IL-15r Agonist (N-803)

Patients with cancer have alterations in immune cell function (40), thus we next sought to determine whether NK cells from patients with chordoma are responsive to ADCC-mediating antibodies and N-803 exposure. We isolated NK cells from three patients with chordoma (CP). Similar to NK cells from healthy donors, NK cells from 3/3 chordoma patients demonstrated enhanced cytotoxicity after exposure to N-601 (FIGS. 5, A and B), cetuximab (FIGS. 5, A and C), N-803 (FIG. 5, A to C), and combination therapy (P≤0.05) (FIG. 5, A to C). This finding indicates that NK cells in chordoma patients are not inherently impaired in their ability to lyse chordoma cells and their cytotoxicity can be enhanced by ADCC and the IL-15 superagonist.

Interestingly, NK cells from ⅔ chordoma patients exhibited significantly increased cytotoxic activity at baseline compared to healthy donor controls, and this persisted after treatment with N-601, cetuximab, N-803, and combination therapy (P≤0.0001). These patients, labeled CP(io), had recurrent chordoma and had undergone various treatment modalities (refer to Materials and Methods) prior to providing blood for this study (FIGS. 5, B and C, and FIG. 10). The patient with NK cell activity similar to that of healthy donors, labeled CP1(n), had a primary chordoma and had not been treated prior to providing blood for the study (FIG. 5A).

### Example 8. PD-L1 t-haNK Cells Induce Lysis of Chordoma Cells

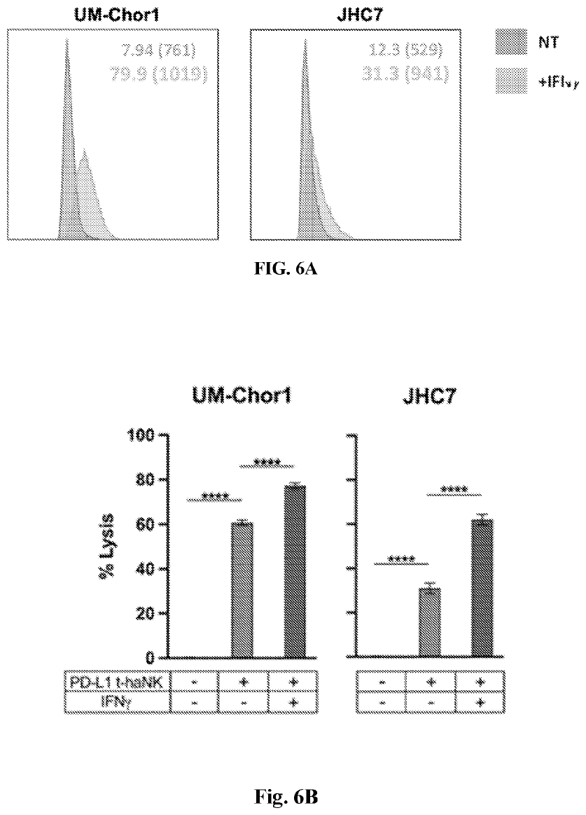

As a potential alternative clinical option for PD-L1 positive tumors, we interrogated the sensitivity of chordoma cell lines to lysis by human NK cells that have been genetically engineered to express the high-affinity CD16 receptor, endoplasmic reticulum-retained IL-2, and a CAR specific against PD-L1 (41, 42). The in vitro anti-tumor efficacy of these PD-L1 targeted high affinity natural killer (PD-L1 t-haNK) cells against human cancer cell lines is dependent on PD-L1 expression levels on tumor cells (20). Here, we hypothesized that treatment of chordoma cell lines with IFNy would upregulate PD-L1 expression, thus increasing their sensitivity to lysis by PD-L1 t-haNK cells. Our study showed that IFNy increased PD-L1 expression (both % positive cells and MFI) on 2/2 chordoma cell lines (FIG. 6A) and in turn, enhanced the susceptibility of these cell lines to lysis by PD-L1 t-haNK cells (P≤0.0001) (FIG. 6B).

Despite containing the high affinity CD16 receptor, it has been shown that tumor cell lysis by PD-L1 t-haNK cells is not enhanced by therapeutic antibodies that induce ADCC, such as avelumab or cetuximab (20). Previous interrogation of this finding revealed that PD-L1 t-haNK killing mediated through the CAR occurs more readily than killing through ADCC (20). Similarly, treatment of PD-L1 t-haNK cells with N-803 does not enhance their cytotoxic function, as this engineered NK cell line does not express the IL-15 receptor (20). Here, we confirm that PD-L1 t-haNK lysis of chordoma cell lines is not enhanced by co-treatment with N601, cetuximab, or N-803 (fig. S3).

### Example 9. Anti-PD-L1 Antibody (N-601)-Mediated ADCC and IL-15/IL-15r Agonist (N-803)-Activation of NK Cells Enhance Lysis of Chordoma CSCs

Cancer stem cells are a subpopulation of cells within certain tumor types that play a significant role in tumorigenesis, metastasis, and resistance to therapy (18, 19, 43) and should therefore be a primary target of anticancer therapy. We have previously reported that exposure of chordoma cells to IFNy increases PD-L1 expression on the cell surface, angmenting anti-PDL1 antibody-mediated ADCC (8). Here, we investigated the susceptibility of chordoma CSCs by N-803-activated NK cells and anti-PD-L1-driven ADCC. Flow cytometry was utilized to distinguish the CSCs from the non-CSCs and to determine the degree of cell death in the two compartments using a viability exclusion dye. As defined in previous studies (8, 22), we designated the UM-Chor1 CSCs as cells that co-express the surface markers CD15, CD24, and CD133 (FIG. 7A). Similar to our data in FIGS. 2A, 4, 5A, and 5C, UM-Chor1 cells were susceptible to N-601-mediated ADCC as evidenced by increased cell death in both CSC and non-CSC populations compared to isotype control (P≤0.05; FIG. 7B). Likewise, activating NK cells with N-803 improved their cytotoxicity against both CSCs and non-CSCs when compared to untreated controls (P≤0.0001). A combinatory approach with N-601 and N-803 further enhanced NK cell lysis of CSC cells (P≤0.0001), while cell death of non-CSCs by N-803 pre-treated NK cells remained the same with or without N-601 (P=0.46) (FIG. 7B).

To elucidate the preferential NK cell killing of UM-Chor1 CSCs over non-CSCs in the presence of N-601 and N-803, we used flow cytometry to examine the expression of NK ligands and PD-L1 on the two cellular subpopulations. CSCs and non-CSCs showed comparable expression of MICA/B, a ligand for the activating receptor NKG2D, and HLA-A,B,C/MHC class I, which binds NK inhibitory receptors. However, CSCs expressed B7-H6, a ligand for the NK activating receptor NKp30, at higher levels than the non-CSCs (>1000% difference in % positive cells and MFI). Furthermore, compared to non-CSCs, the CSCs demonstrated elevated expression of PD-L1, both as % positive cells and MFI (>1000% difference in % positive cells, >20% difference in MFI). This upregulation of an NK-activating ligand and PD-L1 in the UM-Chor1 CSC population may sensitize CSCs to lysis by NK cells in a combinatory treatment paradigm with N-601 and N-803.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Furthermore, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, and the like.

### Informal Sequence Listing

### Illustrative Sequences:

SEQ ID NO: 1. Low Affinity Immunoglobulin Gamma Fc Region Receptor III-A amino acid
sequence (mature form). The phenylalanine at position 158 is underlined.
Arg Thr Glu Asp Leu Pro Lys Ala Val Val Phe Leu Glu Pro Gln Trp Tyr Arg Val Leu Glu Lys
Asp Ser Val Thr Leu Lys Cys Gln Gly Ala Tyr Ser Pro Glu Asp Asn Ser Thr Gln Trp Phe His
Asn Glu Ser Leu Ile Ser Ser Gln Ala Ser Ser Tyr Phe Ile Asp Ala Ala Thr Val Asp Asp Ser
Gly Glu Tyr Arg Cys Gln Thr Asn Leu Ser Thr Leu Ser Asp Pro Val Gln Leu Glu Val His Ile
Gly Trp Leu Leu Leu Gln Ala Pro Arg Trp Val Phe Lys Glu Glu Asp Pro Ile His Leu Arg Cys
His Ser Trp Lys Asn Thr Ala Leu His Lys Val Thr Tyr Leu Gln Asn Gly Lys Gly Arg Lys Tyr
Phe His His Asn Ser Asp Phe Tyr Ile Pro Lys Ala Thr Leu Lys Asp Ser Gly Ser Tyr Phe Cys
Arg Gly Leu Phe Gly Ser Lys Asn Val Ser Ser Glu Thr Val Asn Ile Thr Ile Thr Gln Gly Leu
Ala Val Ser Thr Ile Ser Ser Phe Phe Pro Pro Gly Tyr Gln Val Ser Phe Cys Leu Val Met Val
Leu Leu Phe Ala Val Asp Thr Gly Leu Tyr Phe Ser Val Lys Thr Asn Ile Arg Ser Ser Thr Arg
Asp Trp Lys Asp His Lys Phe Lys Trp Arg Lys Asp Pro Gln Asp Lys
SEQ ID NO: 2. High Affinity Variant F158V Immunoglobulin Gamma Fc Region Receptor
III-A amino acid sequence (mature form). The valine at position 158 is underlined
Arg Thr Glu Asp Leu Pro Lys Ala Val Val Phe Leu Glu Pro Gln Trp Tyr Arg Val Leu Glu Lys
Asp Ser Val Thr Leu Lys Cys Gln Gly Ala Tyr Ser Pro Glu Asp Asn Ser Thr Gln Trp Phe His
Asn Glu Ser Leu Ile Ser Ser Gln Ala Ser Ser Tyr Phe Ile Asp Ala Ala Thr Val Asp Asp Ser
Gly Glu Tyr Arg Cys Gln Thr Asn Leu Ser Thr Leu Ser Asp Pro Val Gln Leu Glu Val His Ile
Gly Trp Leu Leu Leu Gln Ala Pro Arg Trp Val Phe Lys Glu Glu Asp Pro Ile His Leu Arg Cys
His Ser Trp Lys Asn Thr Ala Leu His Lys Val Thr Tyr Leu Gln Asn Gly Lys Gly Arg Lys Tyr
Phe His His Asn Ser Asp Phe Tyr Ile Pro Lys Ala Thr Leu Lys Asp Ser Gly Ser Tyr Phe Cys
Arg Gly Leu Val Gly Ser Lys Asn Val Ser Ser Glu Thr Val Asn Ile Thr Ile Thr Gln Gly Leu
Ala Val Ser Thr Ile Ser Ser Phe Phe Pro Pro Gly Tyr Gln Val Ser Phe Cys Leu Val Met Val
Leu Leu Phe Ala Val Asp Thr Gly Leu Tyr Phe Ser Val Lys Thr Asn Ile Arg Ser Ser Thr Arg
Asp Trp Lys Asp His Lys Phe Lys Trp Arg Lys Asp Pro Gln Asp Lys
SEQ ID NO: 3. Wild-Type IL-2
Met Tyr Arg Met Gln Leu Leu Ser Cys Ile Ala Leu Ser Leu Ala Leu Val Thr Asn Ser Ala Pro
Thr Ser Ser Ser Thr Lys Lys Thr Gln Leu Gln Leu Glu His Leu Leu Leu Asp Leu Gln Met Ile
Leu Asn Gly Ile Asn Asn Tyr Lys Asn Pro Lys Leu Thr Arg Met Leu Thr Phe Lys Phe Tyr
Met Pro Lys Lys Ala Thr Glu Leu Lys His Leu Gln Cys Leu Glu Glu Glu Leu Lys Pro Leu
Glu Glu Val Leu Asn Leu Ala Gln Ser Lys Asn Phe His Leu Arg Pro Arg Asp Leu Ile Ser Asn
Ile Asn Val Ile Val Leu Glu Leu Lys Gly Ser Glu Thr Thr Phe Met Cys Glu Tyr Ala Asp Glu
Thr Ala Thr Ile Val Glu Phe Leu Asn Arg Trp Ile Thr Phe Cys Gln Ser Ile Ile Ser Thr Leu Thr
SEQ ID NO: 4. erIL-2
Met Tyr Arg Met Gln Leu Leu Ser Cys Ile Ala Leu Ser Leu Ala Leu Val Thr Asn Ser Ala Pro
Thr Ser Ser Ser Thr Lys Lys Thr Gln Leu Gln Leu Glu His Leu Leu Leu Asp Leu Gln Met Ile
Leu Asn Gly Ile Asn Asn Tyr Lys Asn Pro Lys Leu Thr Arg Met Leu Thr Phe Lys Phe Tyr
Met Pro Lys Lys Ala Thr Glu Leu Lys His Leu Gln Cys Leu Glu Glu Glu Leu Lys Pro Leu
Glu Glu Val Leu Asn Leu Ala Gln Ser Lys Asn Phe His Leu Arg Pro Arg Asp Leu Ile Ser Asn
Ile Asn Val Ile Val Leu Glu Leu Lys Gly Ser Glu Thr Thr Phe Met Cys Glu Tyr Ala Asp Glu
Thr Ala Thr Ile Val Glu Phe Leu Asn Arg Trp Ile Thr Phe Cys Gln Ser Ile Ile Ser Thr Leu Thr
Gly Ser Glu Lys Asp Glu Leu
SEQ ID NO: 5 PD-L1 scFv amino acid sequence
MDWIWRILFLVGAATGAHSAQPANIQMTQSPSSVSASVGDRVTITCRASQDISRWLA
WYQQKPGKAPKLLIYAASSLQSGVPSRFSGSGSGTDFALTISSLQPEDFATYYCQQAD
SRFSITFGQGTRLEIKGGGGSGGGGSGGGGSGGGGSEVQLVQSGGGLVQPGGSLRLS
CAASGFTFSSYSMNWVRQAPGKGLEWVSYISSSSSTIQYADSVKGRFTISRDNAKNS
LYLQMNSLRDEDTAVYYCARGDYYYGMDVWGQGTTVTVSS
SEQ ID NO: 6. EGFR scFv Protein sequence:
MDWIWRILFLVGAATGAHSAQPADILLTQSPVILSVSPGERVSFSCRASQSIGTNIHW
YQQRTNGSPRLLIKYASESISGIPSRFSGSGSGTDFTLSINSVESEDIADYYCQQNNNW
PTTFGAGTKLELKGGGGSGGGGSGGGGSGGGGSQVQLKQSGPGLVQPSQSLSITCTV
SGFSLTNYGVHWVRQSPGKGLEWLGVIWSGGNTDYNTPFTSRLSINKDNSKSQVFF
KMNSLQSNDTAIYYCARALTYYDYEFAYWGQGTLVTVSA
SEQ ID NO: 7. CD19 scFv-Protein sequence:
MDWIWRILFLVGAATGAHSAQPADIQMTQTTSSLSASLGDRVTISCRASQDISKYLN
WYQQKPDGTVKLLIYHTSRLHSGVPSRFSGSGSGTDYSLTISNLEQEDIATYFCQQGN
TLPYTFGGGTKLELKRGGGGSGGGGSGGGGSGGGGSEVQLQQSGPGLVAPSQSLSV
TCTVSGVSLPDYGVSWIRQPPRKGLEWLGVIWGSETTYYNSALKSRLTIIKDNSKSQ
VFLKMNSLQTDDTAIYYCAKHYYYGGSYAMDYWGQGTTVTVSS
SEQ ID NO: 8. CD20 scFv-Protein sequence:
MDWIWRILFLVGAATGAHSAQPAMAQVKLQESGAELVKPGASVKMSCKASGYTFT
SYNMHWVKQTPGQGLEWIGAIYPGNGDTSYNQKFKGKATLTADKSSSTAYMQLSS
LTSEDSADYYCARSNYYGSSYWFFDVWGQGTTVTVSSGGGGSGGGGSGGGGSDIEL
TQSPTILSASPGEKVTMTCRASSSVNYMDWYQKKPGSSPKPWIYATSNLASGVPARF
SGSGSGTSYSLTISRVEAEDAATYYCQQWSFNPPTFGGGTKLEIKR
SEQ ID NO: 9. CD33 scfV-Protein sequence:
MDWIWRILFLVGAATGAHSAQPADIQMTQSPSSLSASVGDRVTITCRASESVDNYGI
SFMNWFQQKPGKAPKLLIYAASNQGSGVPSRFSGSGSGTDFTLTISSLQPDDFATYYC
QQSKEVPWTFGQGTKVEIKGGGGSGGGGSGGGGSQVQLVQSGAEVKKPGSSVKVS
CKASGYTFTDYNMHWVRQAPGQGLEWIGYIYPYNGGTGYNQKFKSKATITADEST
NTAYMELSSLRSEDTAVYYCARGRPAMDYWGQGTLVTVSS
SEQ ID NO: 10. CSPG4 scfV-Protein sequence:
MDWIWRILFLVGAATGAHSAQPADIELTQSPKFMSTSVGDRVSVTCKASQNVDTNV
AWYQQKPGQSPEPLLFSASYRYTGVPDRFTGSGSGTDFTLTISNVQSEDLAEYFCQQ
YNSYPLTFGGGTKLEIKRAAAEGGGGSGGGGSGGGGSGGGGSAMAQVKLQQSGGG
LVQPGGSMKLSCVVSGFTFSNYWMNWVRQSPEKGLEWIAEIRLKSNNFGRYYAESV
KGRFTISRDDSKSSAYLQMINLRAEDTGIYYCTSYGNYVGHYFDHWGQGTTVTVSS
SEQ ID NO: 11. EGFR scFv-Protein sequence:
MDWIWRILFLVGAATGAHSAQPADILLTQSPVILSVSPGERVSFSCRASQSIGTNIHW
YQQRTNGSPRLLIKYASESISGIPSRFSGSGSGTDFTLSINSVESEDIADYYCQQNNNW
PTTFGAGTKLELKGGGGSGGGGGGGGSGGGGSQVQLKQSGPGLVQPSQSLSITCTV
SGFSLTNYGVHWVRQSPGKGLEWLGVIWSGGNTDYNTPFTSRLSINKDNSKSQVFF
KMNSLQSNDTAIYYCARALTYYDYEFAYWGQGTLVTVSA
SEQ ID NO: 12. IGF1R scFv-Protein sequence:
MDWIWRILFLVGAATGAHSAQPADVVMTQSPLSLPVTPGEPASISCRSSQSLLHSNG
YNYLDWYLQKPGQSPQLLIYLGSNRASGVPDRFSGSGSGTDFTLKISRVEAEDVGVY
YCMQGTHWPLTFGQGTKVEIKGGGGSGGGGSGGGGSGGGGSQVQLQESGPGLVKP
SGTLSLTCAVSGGSISSSNWWSWVRQPPGKGLEWIGEIYHSGSTNYNPSLKSRVTISV
DKSKNQFSLKLSSVTAADTAVYYCARWTGRTDAFDIWGQGTMVTVSS
SEQ ID NO: 13. CD30 scFv-Protein sequence:
MDWIWRILFLVGAATGAHSAQPADIQMTQSPSSLSASVGDRVTITCQASQDISNYLN
WYQQKPGKAPKLLIYDVSNLETGVPSRFSGSGSGTDFTFTISSLQPEDIATYYCQQVA
NVPLTFGQGTKVEIKGGGGSGGGGSGGGGSGGGGSEVQLVESGGGLVQPGGSLRLS
CAASGFTFSSYWMHWVRQAPGKGLEWVSAISWSGDSTYYADSVKGRFTISRDNSK
NTLYLQMNSLRAEDTAVYYCARDRSATWYYLGLGFDVWGQGTLVTVSS
SEQ ID NO: 14. HER2/neu scFv-Protein sequence:
MDWIWRILFLVGAATGAHSAQPADIQMTQSPSSLSASVGDRVTITCRASQDVNTAV
AWYQQKPGKAPKLLIYSASFLYSGVPSRFSGSRSGTDFTLTISSLQPEDFATYYCQQH
YTTPPTFGQGTKVEIKSSGGGGSGGGGSGGGGSGGGGSGGSEVQLVESGGGLVQPG
GSLRLSCAASGFNIKDTYIHWVRQAPGKGLEWVARIYPTNGYTRYADSVKGRFTISA
DTSKNTAYLQMNSLRAEDTAVYYCSRWGGDGFYAMDYWGQGTLVTVSS
SEQ ID NO: 15. GD2 scFv-Protein sequence VL/VH format:
MDWIWRILFLVGAATGAHSAQPASIVMTQTPKFLLVSAGDRVTITCKASQSVSNDVA
WYQQKPGQSPKLLIYSASNRYTGVPDRFTGSGYGTDFTFTISTVQAEDLAVYFCQQD
YSSLGGGTKLEIKGGGGSGGGGSGGGGSGGGGSQVQVKESGPGLVAPSQSLSITCTV
SGFSLTNYGVHWVRQPPGKGLEWLGVIWAGGSTNYNSALMSRLSISKDNSKSQVFL
KMNSLQTDDTAMYYCASRGGNYGYALDYWGQGTSVTVSS
SEQ ID NO: 16. GD2 scFv-Protein sequence VH/VL format:
MDWIWRILFLVGAATGAHSAQPAQVQVKESGPGLVAPSQSLSITCTVSGFSLTNYGV
HWVRQPPGKGLEWLGVIWAGGSTNYNSALMSRLSISKDNSKSQVFLKMNSLQTDD
TAMYYCASRGGNYGYALDYWGQGTSVTVSSGGGGSGGGGSGGGGSGGGGSSIVM
TQTPKFLLVSAGDRVTITCKASQSVSNDVAWYQQKPGQSPKLLIYSASNRYTGVPDR
FTGSGYGTDFTFTISTVQAEDLAVYFCQQDYSSLGGGTKLEIK
SEQ ID NO: 17. CD123 scFv Protein sequence:
MDWIWRILFLVGAATGAHSAQPALPVLTQSASVSGSPGQSITISCTGTSSDVGRYDY
VSWYQQHPGKAPQLMIYDVSNRPSGVSNRFSGSKSGNTASLTISGLQAEDEADYYCS
SYTGSSTLYVFGTGTKVTVLGQPKGGGGSGGGGSGGGGSEVQLVESGGGLVKPGGS
LRLSCAASGFTFSNAWMSWVRQAPGKGLEWVGRIKSKTDGGTTDYAAPVKGRFTIS
RDDSKNTLYLQMNSLKTEDTAVYYCTTDYDFWSGYYYWGQGTLVTVSS
SEQ ID NO: 18. B7-H4 scFv Protein sequence:
MDWIWRILFLVGAATGAHSAQPAEVQLVESGGGLVQPGGSLRLSCAASGFTFNSYA
MHWVRQAPGKGLEWVSAISGNGGSTRYADSVKGRFTISRDNSKNTLYLQMNSLRA
EDTAVYYCARDRFRKVHGFDVWGQGTLVTVSSGGGGSGGGGSGGGGSGGGGDIQ
MTQSPSSLSASVGDRVTITCQASQDISNYLNWYQQKPGKAPKLLIYDASNLETGVPS
RFSGSGSGTDFTFTISSLQPEDIATYYCQQDATFPLTFGQGTKVEIK
SEQ ID NO: 19. FAP scFv Protein sequence VL/VH format:
MDWIWRILFLVGAATGAHSAQPADIVMTQSPSSLAVSVGEKVTMSCKSSQSLLYSR
NQKNYLAWFQQKPGQSPKLLIFWASTRESGVPDRFTGSGFGTDFNLTISSVQAEDLA
VYDCQQYFSYPLTFGAGTKLELRGGGGSGGGGSGGGGSGGGGSQVQLQQSGPELV
KPGASVKMSCKTSRYTFTEYTIHWVRQSHGKSLEWIGGINPNNGIPNYNQKFKGRAT
LTVGKSSSTAYMELRSLTSEDSAVYFCARRRIAYGYDEGHAMDYWGQGTSVTVSS
SEQ ID NO: 20. FAP scFv Protein sequence VH/VL format:
MDWIWRILFLVGAATGAHSAQPAQVQLQQSGPELVKPGASVKMSCKTSRYTFTEYT
IHWVRQSHGKSLEWIGGINPNNGIPNYNQKFKGRATLTVGKSSSTAYMELRSLTSED
SAVYFCARRRIAYGYDEGHAMDYWGQGTSVTVSSGGGGSGGGGSGGGGSGGGGS
DIVMTQSPSSLAVSVGEKVTMSCKSSQSLLYSRNQKNYLAWFQQKPGQSPKLLIFWA
STRESGVPDRFTGSGFGTDFNLTISSVQAEDLAVYDCQQYFSYPLTFGAGTKLELR
SEQ ID NO: 21. CD8 Hinge Region
KPTTTPA PRPPTPAPTI ASQPLSLRPE ACRPAAGGAV HTRGLDFACF WVLVVVG
SEQ ID NO: 22. CD28 Transmembrane domain
GVL ACYSLLVTVA FIIFWVR
SEQ ID NO: 23. CD28 signaling domain
RSKRSRLLHSDYMNMTPRRPGPTRKHYQPYAPPRDFAAYRS
SEQ ID NO: 24. 4-1BB signaling domain
KRGRKKLLYIFKQPFMRPVQTTQEEDGCSCRFPEEEEGGCEL
SEQ ID NO: 25. CD3ζ signaling domain
RVKFSRSADAPAYQQGQNQLYNELNLGRREEYDVLDKRRGRDPEMGGKPQRRKNP
QEGLYNELQKDKMAEAYSEIGMKGERRRGKGHDGLYQGLSTATKDTYDALHMQA
LPPR
SEQ ID NO: 26 the nucleic acid sequence that encodes PD-L1 CAR
ATGGACTGGATCTGGCGGA TTCTGTTTCT
CGTGGGAGCTGCCACAGGCGCTCATTCTGC TCAGCCTGCCAACATCCAGA
TGACCCAGTC TCCATCTTCT GTGTCTGCAT CTGTAGGAGA
CAGAGTCACCATCACTTGTC GGGCGAGTCA GGATATTAGC CGCTGGTTAG
CCTGGTATCA GCAGAAACCAGGGAAAGCCC CTAAACTCCT GATCTATGCT
GCATCCAGTT TGCAAAGTGG GGTCCCATCGAGGTTCAGCG GCAGTGGATC
TGGGACAGAT TTCGCTCTCA CTATCAGCAG CCTGCAGCCTGAAGATTTTG
CAACTTACTA TTGTCAACAG GCTGACAGTC GTTTCTCGAT
CACCTTCGGCCAAGGGACAC GACTGGAGAT TAAAGGCGGC GGAGGAAGCG
GAGGCGGAGG ATCTGGGGGCGGAGGCTCTG GCGGAGGGGG ATCTGAGGTG
CAGCTGGTGC AGTCTGGGGG AGGCTTGGTACAGCCTGGGG GGTCCCTGAG
ACTCTCCTGT GCAGCCTCTG GATTCACCTT CAGTAGCTATAGCATGAACT
GGGTCCGCCA GGCTCCAGGG AAGGGGCTGG AGTGGGTTTC
ATACATTAGTAGTAGTAGTA GTACCATACA GTACGCAGAC TCTGTGAAGG
GCCGATTCAC CATCTCCAGAGACAATGCCA AGAACTCACT GTATCTGCAA
ATGAACAGCC TGAGAGACGA GGACACGGCTGTGTATTACT GTGCGAGAGG
GGACTACTAC TACGGTATGG ACGTCTGGGG CCAAGGGACCACGGTCACCG
TGAGCTCAGC GGCCGCGCTG AGCAACAGCA TCATGTACTT
CAGCCACTTCGTGCCTGTGT TCCTGCCTGC CAAGCCTACA ACAACACCAG
CCCCTAGACC TCCAACCCCTGCCCCTACAATTGCCTCTCA GCCTCTGTCT
CTGAGGCCCG AAGCTTGTAG ACCTGCTGCTGGCGGAGCTG TGCACACCAG
AGGACTGGAT TTCGCCTGCT TTTGGGTGCT GGTGGTCGTGGGCGGAGTGC
TGGCTTGTTA TTCTCTGCTG GTCACCGTGG CCTTCATCAT
CTTTTGGGTCCGACTGAAGA TCCAGGTCCG AAAGGCCGCC ATCACCAGCT
ACGAGAAGTC TGATGGCGTGTACACCGGCC TGAGCACCAG AAACCAGGAA
ACCTACGAGA CACTGAAGCA CGAGAAGCCCCCCCAG
SEQ ID NO: 27 the amino acid sequence of PD-L1 CAR
MDWIWRILFL VGAATGAHSA QPANIQMTQS PSSVSASVGD RVTITCRASQ
DISRWLAWYQQKPGKAPKLL IYAASSLQSG VPSRFSGSGS GTDFALTISS
LQPEDFATYY CQQADSRFSITFGQGTRLEI KGGGGSGGGG SGGGGSGGGG
SEVQLVQSGG GLVQPGGSLR LSCAASGFTFSSYSMNWVRQ APGKGLEWVS
YISSSSSTIQ YADSVKGRFT ISRDNAKNSL YLQMNSLRDEDTAVYYCARG
DYYYGMDVWG QGTTVTVSSA AALSNSIMYF SHFVPVFLPA
KPTTTPAPRPPTPAPTIASQ PLSLRPEACR PAAGGAVHTR GLDFACFWVL
VVVGGVLACY SLLVTVAFIIFWVRLKIQVR KAAITSYEKS DGVYTGLSTR
NQETYETLKH EKPPQ

1. A method of treating chordoma, the method comprising administering to a subject in need thereof a plurality of haNK cells at a dosage effective to treat the chordoma, wherein the haNK cells each comprise a first chimeric antigen receptor (CAR) comprising a means for binding PD-L1 and a second CAR comprising an amino acid sequence having at least 85% identity to a sequence selected from the group consisting of SEQ ID NOs: 6-20.

2. The method of claim 1, wherein the haNK cells preferentially target cancer stem cells to non-cancer stem cells in the chordoma.

3. The method of claim 2, wherein the cancer stem cells co-express surface markers CD15, CD24, and CD133.

4. A method of claim 1, wherein the method further comprises administering an anti-EGFR antibody.

5. The method of claim 4, wherein the anti-EGFR antibody is cetuximab.

6. The method of claim 5, wherein the anti-EGFR antibody is administered at a dosage from 100 mg/m² to 1000 mg/m².

7. The method of claim 4, wherein the anti-EGFR antibody is co-administered at the same time as the haNK cells.

8. The method of claim 1, wherein the haNK cells are administered at a dosage from 5×10⁵ cells/kg to 5×10⁸ cells/kg.

9. The method of claim 1, wherein the haNK cells further express recombinant IL2.

10. The method of claim 1, wherein the CAR comprises an amino acid sequence having at least 95% identity to a sequence selected from the group consisting of SEQ ID NOs: 6-20.

11. The method of claim 1, wherein the haNK cells are irradiated before administration at a radiation dose of at least 500 cGy.

12. A method of treating chordoma comprising: stimulating NK cells with an IL-15 superagonist; and administering to a subject in need thereof the NK cells and a means for binding PD-L1 and/or a means for binding EGFR.

13. The method of claim 12, wherein the NK cells preferentially target cancer stem cells to non-cancer stem cells in the chordoma.

14. The method of claim 12, wherein the NK cells are stimulated with N-803.

15. The method of claim 12, wherein the NK cells are autologous NK cells.

16. The method of claim 1, further comprising administering a further cancer treatment to the subject.

17. The method of claim 16, wherein the further cancer treatment comprises a chemotherapy.

18. The method of claim 17, wherein the chemotherapy comprises at least one of aldoxorubicin, cyclophosphamide, irinotecan, gemcitabine, capecitabine, 5-FU, FOLFIRI, FOLFOX, and oxiplatin.

19. The method of claim 16, wherein the further cancer treatment comprises a radiotherapy.

20. The method of claim 12, wherein the means for binding PD-L1 is a monoclonal antibody.

20. The method of claim 12, wherein the means for binding EGFR is a monoclonal antibody.

21. A kit for treating chordoma in a subject in need thereof, the kit comprising (i) NK cells and (ii) means for binding PD-L1 and/or a means for binding EGFR.

22. The kit of claim 21, further comprising an IL-15 or an IL-15 superagonist.

23. The kit of claim 21 wherein the means for binding PD-L1, or the means for binding EGFR antibody, or both are monoclonal antibodies.

24. The kit of claim 23, wherein the means for binding PD-L1 is an anti PD-L1 antibody, and wherein the anti PD-L1 antibody is a humanized non-human anti-PD-L1 antibody.

25. The kit of claim 23, wherein the EGFR antibody is cetuximab.


SEQUENCE LISTING (27 sequences)

SEQ ID NO: 1 - protein, 236 aa, synthetic construct
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 2 - protein, 236 aa, synthetic construct
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 160 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 5 - protein, 269 aa, synthetic construct
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSS

SEQ ID NO: 6 - protein, 269 aa, synthetic construct
MDWIWRILFLVGAATGAHSAQPADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 7 - protein, 271 aa, synthetic construct
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 8 - protein, 269 aa, synthetic construct
MDWIWRILFLVGAATGAHSAQPAMAQVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKR

SEQ ID NO: 9 - protein, 265 aa, synthetic construct
MDWIWRILFLVGAATGAHSAQPADIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 10 - protein, 280 aa, synthetic construct
MDWIWRILFLVGAATGAHSAQPADIELTQSPKFMSTSVGDRVSVTCKASQNVDTNVAWYQQKPGQSPEPLLFSASYRYTGVPDRFTGSGSGTDFTLTISNVQSEDLAEYFCQQYNSYPLTFGGGTKLEIKRAAAEGGGGSGGGGSGGGGSGGGGSAMAQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 11 - protein, 269 aa, synthetic construct; identical to SEQ ID NO: 6

SEQ ID NO: 12 - protein, 274 aa, synthetic construct
MDWIWRILFLVGAATGAHSAQPADVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKGGGGSGGGGSGGGGSGGGGSQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSS

SEQ ID NO: 13 - protein, 274 aa, synthetic construct
MDWIWRILFLVGAATGAHSAQPADIQMTQSPSSLSASVGDRVTITCQASQDISNYLNWYQQKPGKAPKLLIYDVSNLETGVPSRFSGSGSGTDFTFTISSLQPEDIATYYCQQVANVPLTFGQGTKVEIKGGGGSGGGGSGGGGSGGGGSEVQLVESGGGLVQPGGSLRLSCAASGFTFSSYWMHWVRQAPGKGLEWVSAISWSGDSTYYADSVKGRFTISRDNSKNTLYLQMNSLRAEDTAVYYCARDRSATWYYLGLGFDVWGQGTLVTVSS

SEQ ID NO: 14 - protein, 275 aa, synthetic construct
MDWIWRILFLVGAATGAHSAQPADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSSGGGGSGGGGSGGGGSGGGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 15 - protein, 266 aa, synthetic construct
MDWIWRILFLVGAATGAHSAQPASIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIKGGGGSGGGGSGGGGSGGGGSQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSS

SEQ ID NO: 16 - protein, 266 aa, synthetic construct
MDWIWRILFLVGAATGAHSAQPAQVQVKESGPGLVAPSQSLSITCTVSGFSLTNYGVHWVRQPPGKGLEWLGVIWAGGSTNYNSALMSRLSISKDNSKSQVFLKMNSLQTDDTAMYYCASRGGNYGYALDYWGQGTSVTVSSGGGGSGGGGSGGGGSGGGGSSIVMTQTPKFLLVSAGDRVTITCKASQSVSNDVAWYQQKPGQSPKLLIYSASNRYTGVPDRFTGSGYGTDFTFTISTVQAEDLAVYFCQQDYSSLGGGTKLEIK

SEQ ID NO: 17 - protein, 274 aa, synthetic construct
MDWIWRILFLVGAATGAHSAQPALPVLTQSASVSGSPGQSITISCTGTSSDVGRYDYVSWYQQHPGKAPQLMIYDVSNRPSGVSNRFSGSKSGNTASLTISGLQAEDEADYYCSSYTGSSTLYVFGTGTKVTVLGQPKGGGGSGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDYDFWSGYYYWGQGTLVTVSS

SEQ ID NO: 18 - protein, 269 aa, synthetic construct
MDWIWRILFLVGAATGAHSAQPAEVQLVESGGGLVQPGGSLRLSCAASGFTFNSYAMHWVRQAPGKGLEWVSAISGNGGSTRYADSVKGRFTISRDNSKNTLYLQMNSLRAEDTAVYYCARDRFRKVHGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQDATFPLTFGQGTKVEIK

SEQ ID NO: 19 - protein, 280 aa, synthetic construct
MDWIWRILFLVGAATGAHSAQPADIVMTQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELRGGGGSGGGGSGGGGSGGGGSQVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSS

SEQ ID NO: 20 - protein, 280 aa, synthetic construct
MDWIWRILFLVGAATGAHSAQPAQVQLQQSGPELVKPGASVKMSCKTSRYTFTEYTIHWVRQSHGKSLEWIGGINPNNGIPNYNQKFKGRATLTVGKSSSTAYMELRSLTSEDSAVYFCARRRIAYGYDEGHAMDYWGQGTSVTVSSGGGGSGGGGSGGGGSGGGGSDIVMTQSPSSLAVSVGEKVTMSCKSSQSLLYSRNQKNYLAWFQQKPGQSPKLLIFWASTRESGVPDRFTGSGFGTDFNLTISSVQAEDLAVYDCQQYFSYPLTFGAGTKLELR

SEQ ID NO: 21 - protein, 54 aa, synthetic construct
KPTTTPAPRPPTPAPTIASQPLSLRPEACRPAAGGAVHTRGLDFACFWVLVVVG

SEQ ID NO: 22 - protein, 20 aa, synthetic construct
GVLACYSLLVTVAFIIFWVR

SEQ ID NO: 23 - protein, 41 aa, synthetic construct
RSKRSRLLHSDYMNMTPRRPGPTRKHYQPYAPPRDFAAYRS

SEQ ID NO: 24 - protein, 42 aa, synthetic construct
KRGRKKLLYIFKQPFMRPVQTTQEEDGCSCRFPEEEEGGCEL

SEQ ID NO: 25 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - DNA, 1215 bp, synthetic construct
  source 1..1215: mol_type = other DNA
atggactggatctggcggattctgtttctcgtgggagctgccacaggcgctcattctgctcagcctgccaacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggcggcggaggaagcggaggcggaggatctgggggcggaggctctggcggagggggatctgaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctcagcggccgcgctgagcaacagcatcatgtacttcagccacttcgtgcctgtgttcctgcctgccaagcctacaacaacaccagcccctagacctccaacccctgcccctacaattgcctctcagcctctgtctctgaggcccgaagcttgtagacctgctgctggcggagctgtgcacaccagaggactggatttcgcctgcttttgggtgctggtggtcgtgggcggagtgctggcttgttattctctgctggtcaccgtggccttcatcatcttttgggtccgactgaagatccaggtccgaaaggccgccatcaccagctacgagaagtctgatggcgtgtacaccggcctgagcaccagaaaccaggaaacctacgagacactgaagcacgagaagcccccccag

SEQ ID NO: 27 - protein, 405 aa, synthetic construct
MDWIWRILFLVGAATGAHSAQPANIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKGGGGSGGGGSGGGGSGGGGSEVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSSAAALSNSIMYFSHFVPVFLPAKPTTTPAPRPPTPAPTIASQPLSLRPEACRPAAGGAVHTRGLDFACFWVLVVVGGVLACYSLLVTVAFIIFWVRLKIQVRKAAITSYEKSDGVYTGLSTRNQETYETLKHEKPPQ